SEQUENCE LISTING (136 sequences)

SEQ ID NO: 1 - DNA, 394 bp, Homo sapiens
tttgttaagatatatatatatttttttttttttaagcactcctttgaaaggattaaggacgcctaacttgaaggaaaagcatttctgcacaggtgtcagtgtattgcactgtggaacctgtgtggtaaaggcaaagggggtagtgcttatctcttgatcctaaatatgtgagaccagattaaagtgaaatctgggaggcaatgaatgttaaatgagttgttatgtaatttgcatagaggtgatgctgagagatttagaaaggatcactgtgggttgcttgctcactttcttgctctcctattccgtagctttccaaatggctgtactcaacggtggcttggtgtttaggggatttaaggggggcaaaaagaaagattaataatctcctcctctc

SEQ ID NO: 2 - DNA, 333 bp, Homo sapiens
agtcttgccaagcagcattgctggtttaggaatttgtgaatttgtatcctgctcattaattctgcagaatggagcagtgcgtgaagagggcttgggggaaaatgcgcccccgtctgagtaggaaggcctgagcccatgtcaaggcagacacatcgtctccctttctgctagggccccttgtggaaccccctacccccgctttagccccacttgaacaacgttcggactttgagcagcgcacactatcctcagctcaccttatccacctcctgaaggccttctgggagttaaaaatggcacttaagctgtaggagaaagcttgttaaccacttt

SEQ ID NO: 3 - DNA, 121 bp, Homo sapiens
tcgtcttgccaagcagcattgctggtttaggaatttgtgcgtcttgtgagtgtgtgtgtgtgggtgtgtgtcgtcttgccaagcagcattgctggtttaggaatttgtgcgtcttgtgaga

SEQ ID NO: 4 - DNA, 373 bp, Homo sapiens
tggggtagtttttatgacctagatcctaaattgttcactgctgctgttgctactcttggtactttttactggctggcatctgcttgcttaagtttataacatagtaggagcattaacaaggtcccacggtggggaccttggtcgtttgacgagatctgcgctcccgcccatcccctcccccccccctccacattggagacgcggccaccaccgcgctggcgcggagagagggaggaccgggcgtcatgctgtttctggcctgaggttttgtgtgcctttgttttccttttgctctattcgtgtattcctgcctacggcctgtgcggggaattaggagctcagtactgaaacggcggttttcctaaacagtacc

SEQ ID NO: 5 - DNA, 203 bp, Homo sapiens
aatcgtttcccagataccttgaacaaaaatccagcagttagagaagcctgaccatgaagcaaatttgacttttgtccctctagataacaaaagttatctttttgaaagtaatggtgtaatttgaatgagtgtagagaagcgctgaagactgagctttactaaagccttcagacctggatttggcagcagcgtggccttagtca

SEQ ID NO: 6 - DNA, 62 bp, Homo sapiens
aacaccatgaaagggcatcagctggagatactgctataaagggactgccttgtaatttcata

SEQ ID NO: 7 - DNA, 170 bp, Homo sapiens
ggggcgtttttagcgcagtgtgcaagtgccctattaggggtaggcgcccagtaactcgagaagcatggagtaggaaaccacaaacagcacctgctccccctcctctccccctacctgctgtggggaaggcctcccttgtaaatttgaaaggttgattcacgggaagccgt

SEQ ID NO: 8 - DNA, 302 bp, Homo sapiens
accaccatcacctaaacgttggtaactggagcagctgctagtgtcagtgcggactaaacaggagacggcgggaaccgtgtccagccagggtcctgggccgcgacctggttctcccggagtctacagtgaggatgacgggcggggagagggggccggcgggacccgcgtgtcccaggcaactccgggagagggagaagcaggggtggctcggcgggggctcggcgggggctcggcgggggctcggcgggggctcggcgggggctcggcgggggctcggcgggggcagcgcccgctgcagggag

SEQ ID NO: 9 - DNA, 490 bp, Homo sapiens
cccagccccagcctccagactggctgagatcacagtgtgcgccaccccatccctggcttgcgatggtctcttactccccactttacaagggaggaaagaggccgagggcagagtgggccgcctgaggtttggaggccagggctagtacaacctcaatttgaccctggaacctgccgcttcccccacccaggtgcgggacccacccgcttgtccacacctggctctgcccaccgcccccggctgctcctctgggctcaggtcgccgcggtcaggagctgccaagtttgcctatcaaactttatcttcgtgcagagaactgcagcctggagctggttattccggtcagtgaaaacgttgcatttctacatatgcttatcatcatctgtgtaaacatttcttggtataactgtggaacagtcagtaaatatagataaatcgaagagtaggtctattgcatatgctataaaaaaatgcttttactatcaaccta

SEQ ID NO: 10 - DNA, 208 bp, Homo sapiens
cacacaacgtcctcaggcacgtatctctttaagaatgtgtcccgagaaggcgtcccaggagtcttgatttttatttagccgtccacggttgcccctttgggtgcttcgccttcagatggggtgaagggctcacttgttagctggctggccccaaagacaggcttgttttccaccaggcagtgactgaagccggcagcctgctgcatac

SEQ ID NO: 11 - DNA, 1077 bp, Homo sapiens
atttaactgcactgaagggaaaacgtgggaaatggatttttggtgctgtgtagaccacatttcatagcggttggcatctcacatgcttatgcaaagcctaactccgcaccctggggcagacagtgggagcccagctggattcctacactcaagccctccagcatcaggtttattttccaggacaccagagtgattgtttattccataattcccacaaaggagacagtaaacaacagaacagaggtggagcgggcacggaaggccaggaagtggatggtggctgccagcacttctgtggagacccagggcccccctccaggagccccggtgttcaacctccacagtgaacaggggatggatggctgagatgtcctcgaaatttttttggacttcctcaggcgaccacaggattccttctcaaagagcctgatctcagcaggcacccgaatgggcatcctggtgcttcatgctctacaacagctgggaacgccatgtcctggccccaggcgactggaaactgctttcctccccgacatcagcaccaaggggaatgttcccagtgccattcttccaagtcagggagagcgtcacaatagaaaccgtctctgtggagaggatggcacctgaagccatggaataggaagggagcatcagaggctgctggctggtcctgcagagccgcctgagaggcctgtgggagcagcagagggtcccggccttgggcgccatccgctctctgctgctctggagggagaaaaaggacaagttgaaacttgcacaagcagcctccattctggggagttcccttgtattccccacaccaacccgcacctcagcgaaggcctgtggaagacttctgcagtgacagccccgatgaaccatgcttgccgtgcccgtcccctgtgcggtgcctcacgtccactcaggccccggccatctcaccctcctggggagatggagggaagcaccatggggatttgctttttcttgctgccgacggagcccagccaccacgggagggaggcccggccagcctgcggtgggtgggtgacatgtggcccggatctgcccggggcg

SEQ ID NO: 12 - DNA, 866 bp, Homo sapiens
tctctcatcaatgaaaactgagctacgatctatcagctcagcacataacaacacaggatcatcctaattatttccaatgtcccccagagtgattcttttttcttatgataaaaattatctggaaatttctaggcacataagcagctgaaaaggttgcatgtgaggcaatgaatagcagaaatatttgtgtgccatttttatatcaacatgttaatgtgtgctaatgaattttagagtggatttttttaaaaacatgactattgtaattaaaagcttatgtgatttaaatacaagatgcaatgatgattaccagattctattcctctttatattttacataagacttccagggttccataagtataatcttgaaacataggacattcctggaaaaaaaaattgcttaaaactatttcagtgtgcaatccacctctttgtaaatgttactttcttccatccaaatattacatttggcaacaaaactctccgctgagagctttctcttccttttgtgtcattgttccctaaataaagcaacacatgaaattcctgacggcaaaaatcagactcagatcccaaaacctctgtctttatgcaagatttatcttttgcattggaaacggccaaggaatatgaagaggggaaagaagaggcaaacagacaagcatgcaggctctgaggaataaatgcccctcaggacgctgtctcctggggaattgcaaacctcagtccgtttctgaggaagtgcggtctctgcatttctgaaagaggtatttcccccccttgacacaaggagcatggtaatgaattgactagttaaaaactgttggttggaaaaacccgtccctgtgtctttctgagcaggg

SEQ ID NO: 13 - DNA, 1650 bp, Homo sapiens
ggtcagtgcccgcacagccgcaaagccacagagagacgtggttttgcctcctgtggtgcttgtgatctatggagagacagacattgaagaaacaagcaaaatatcacacaggagacctgtaacattgtaggactgatgagagcaacagtgagttgggggagctttaagcaactccaacaagaggaccctttctggccttgattgagaaaactcttcctcctggcacacacatgccatttgggtgtggggactcactgcggaggcagcactgccccttctatccagccaagggctcctttctatgcctcacccgtctccccccgacccctataggttcatgccgggctgcagttggctttgtagctgcctgttcctccttgcaacctctctgctggtgctttgttttgtgggaagagcagggactgagcatgaagtagctggggacaccacagcgacatctcctgatgggcaccgggcgagattggctcctgctcctgaagcctcttccaactgctgctcatcccaccacgttgcaaagctgcgctgaggcactgtgggaaacaggcaggaggtgaggaggctcatcggggaatggcagcttaccagcactgctcactcgcagccctgcttgcaacagcagagtgagacgtgctggaaaccacgctggagttaacgcacttgtcgctgcgagcctcttctttaaagcccctctgtgatgagaaacaaaggctttctcgcctagccgtcgccctagcatcatctattatgcatcgtcttatgcaaaataaataaaatacagaaaacagcaagtatctgtgaaaacagcattttcataaattatacaaaaaagaaccaattggaggaacttcaagaaaaaggtttgccaaaatttcgatcgtaactttcaaattagggtctaaatgaagtgttcaaagacagttttcagttttgtttccccactccaccaacatcagaagaacatttaacagacacgttactttccggccaccatacctgcccttggagtgaaacaagggttcatgtatacgagtctcccagacagcacggttgctccgtgtgatgatggagctattggggccattaataaatgctgaccaccttgtgggggctgtgtgtgtgtgtgaagctcttctgaagcaccttccttcggtgactccaccccctgatgggttttctgcagatacagaaatagggcaactctggattagtgcagcacattgtgtgaagctcagaagtttcacaggcagagtcctagagctgtgtttctcgatatagtttaagcactcaaatatctcgggtattgcagagaggaagtgagagagctatgtgccttctcaaccctcgctgggaattatcagcagagctgagctgtgctgtcatgggaacagggttgttgaagccagccctgaagagggatgttggtttgattgtggtcctcagtctgagttggactagaaggttcaacattctaaaaggaaaacgcagaagccctgtctttctgaggcttgtctgcaacatacgctcattccttccaaattatttgctggaaaaaatttaataaataatcatagagtaattaacatgttccagttcctgtctatactggttgatactgattcaacgagtatg

SEQ ID NO: 14 - RNA, 21 nt, Artificial Sequence
  Exemplified ADAR1p110 aRNA Antisense Sequence
gcaagacgacacacacccauu

SEQ ID NO: 15 - RNA, 21 nt, Artificial Sequence
  Exemplified ADAR1p110 aRNA Antisense Sequence
ugcuuggcaagacgacacauu

SEQ ID NO: 16 - RNA, 21 nt, Artificial Sequence
  Exemplified ADAR1p110 aRNA Antisense Sequence
acggaauaggagagcaagauu

SEQ ID NO: 17 - RNA, 21 nt, Artificial Sequence
  Exemplified ADAR1p110 aRNA Antisense Sequence
caaguuaggcguccuuaauuu

SEQ ID NO: 18 - RNA, 21 nt, Artificial Sequence
  Exemplified ADAR1p110 aRNA Antisense Sequence
gcuuucuccuacagcuuaauu

SEQ ID NO: 19 - RNA, 21 nt, Artificial Sequence
  Exemplified ADAR1p110 aRNA Antisense Sequence
cuuucaaaggagugcuuaauu

SEQ ID NO: 20 - RNA, 21 nt, Artificial Sequence
  Exemplified ADAR1p110 aRNA Antisense Sequence
augcugcuuggcaagacgauu

SEQ ID NO: 21 - RNA, 21 nt, Artificial Sequence
  Exemplified ADAR1p110 aRNA Antisense Sequence
cuagcagaaagggagacgauu

SEQ ID NO: 22 - RNA, 21 nt, Artificial Sequence
  Exemplified ADAR1p110 aRNA Antisense Sequence
aaagugagcaagcaacccauu

SEQ ID NO: 23 - RNA, 21 nt, Artificial Sequence
  Exemplified ADAR1p110 aRNA Antisense Sequence
aguccgaacguuguucaaguu

SEQ ID NO: 24 - RNA, 21 nt, Artificial Sequence
  Exemplified ADAR1p110 aRNA Antisense Sequence
aggauacaaauucacaaauuu

SEQ ID NO: 25 - RNA, 21 nt, Artificial Sequence
  Exemplified ADAR1p110 aRNA Antisense Sequence
aaguuaggcguccuuaaucuu

SEQ ID NO: 26 - RNA, 21 nt, Artificial Sequence
  Exemplified ADAR1p110 aRNA Antisense Sequence
caccugugcagaaaugcuuuu

SEQ ID NO: 27 - RNA, 21 nt, Artificial Sequence
  Exemplified ADAR1p110 aRNA Antisense Sequence
uuaccacacagguuccacauu

SEQ ID NO: 28 - RNA, 21 nt, Artificial Sequence
  Exemplified ADAR1p110 aRNA Antisense Sequence
caagagauaagcacuacccuu

SEQ ID NO: 29 - RNA, 21 nt, Artificial Sequence
  Exemplified ADAR1p110 aRNA Antisense Sequence
uaaauccccuaaacaccaauu

SEQ ID NO: 30 - RNA, 21 nt, Artificial Sequence
  Exemplified ADAR1p110 aRNA Antisense Sequence
uuaaauccccuaaacaccauu

SEQ ID NO: 31 - RNA, 21 nt, Artificial Sequence
  Exemplified ADAR1p110 aRNA Antisense Sequence
uuucuccuacagcuuaaguuu

SEQ ID NO: 32 - RNA, 21 nt, Artificial Sequence
  Exemplified ADAR1p110 aRNA Antisense Sequence
acucauuuaacauucauuguu

SEQ ID NO: 33 - RNA, 21 nt, Artificial Sequence
  Exemplified ADAR1p110 aRNA Antisense Sequence
ucuccuacagcuuaagugcuu

SEQ ID NO: 34 - RNA, 21 nt, Artificial Sequence
  Exemplified ADAR1p110 aRNA Antisense Sequence
cuuaaauccccuaaacaccuu

SEQ ID NO: 35 - RNA, 21 nt, Artificial Sequence
  Exemplified ADAR1p110 aRNA Antisense Sequence
uucuccuacagcuuaaguguu

SEQ ID NO: 36 - RNA, 21 nt, Artificial Sequence
  Exemplified ADAR1p110 aRNA Antisense Sequence
auaagcacuacccccuuuguu

SEQ ID NO: 37 - RNA, 21 nt, Artificial Sequence
  Exemplified ADAR1p150 aRNA Antisense Sequence
aaggcagucccuuuauagcuu

SEQ ID NO: 38 - RNA, 21 nt, Artificial Sequence
  Exemplified ADAR1p150 aRNA Antisense Sequence
uauagcaguaucuccagcuuu

SEQ ID NO: 39 - RNA, 21 nt, Artificial Sequence
  Exemplified ADAR1p150 aRNA Antisense Sequence
uucagcgcuucucuacacuuu

SEQ ID NO: 40 - RNA, 21 nt, Artificial Sequence
  Exemplified ADAR1p150 aRNA Antisense Sequence
uccuacuccaugcuucucguu

SEQ ID NO: 41 - RNA, 21 nt, Artificial Sequence
  Exemplified ADAR1p150 aRNA Antisense Sequence
uuuuuguucaagguaucuguu

SEQ ID NO: 42 - RNA, 21 nt, Artificial Sequence
  Exemplified ADAR1p150 aRNA Antisense Sequence
uaauagggcacuugcacacuu

SEQ ID NO: 43 - RNA, 21 nt, Artificial Sequence
  Exemplified ADAR1p150 aRNA Antisense Sequence
uuauagcaguaucuccagcuu

SEQ ID NO: 44 - RNA, 21 nt, Artificial Sequence
  Exemplified ADAR1p150 aRNA Antisense Sequence
uuacugggcgccuaccccuuu

SEQ ID NO: 45 - RNA, 21 nt, Artificial Sequence
  Exemplified ADAR1p150 aRNA Antisense Sequence
uguuugugguuuccuacucuu

SEQ ID NO: 46 - RNA, 21 nt, Artificial Sequence
  Exemplified ADAR1p150 aRNA Antisense Sequence
uuuauagcaguaucuccaguu

SEQ ID NO: 47 - RNA, 21 nt, Artificial Sequence
  Exemplified ADAR1p150 aRNA Antisense Sequence
aguacugagcuccuaauucuu

SEQ ID NO: 48 - RNA, 21 nt, Artificial Sequence
  Exemplified ADAR1p150 aRNA Antisense Sequence
uuuuguucaagguaucugguu

SEQ ID NO: 49 - RNA, 21 nt, Artificial Sequence
  Exemplified ADAR1p150 aRNA Antisense Sequence
uuaagcaagcagaugccaguu

SEQ ID NO: 50 - RNA, 21 nt, Artificial Sequence
  Exemplified ADAR1p150 aRNA Antisense Sequence
uucaaauuacaccauuacuuu

SEQ ID NO: 51 - RNA, 21 nt, Artificial Sequence
  Exemplified ADAR1p150 aRNA Antisense Sequence
cuacuauguuauaaacuuauu

SEQ ID NO: 52 - RNA, 21 nt, Artificial Sequence
  Exemplified ADAR1p150 aRNA Antisense Sequence
ggauuuuuguucaagguauuu

SEQ ID NO: 53 - RNA, 21 nt, Artificial Sequence
  Exemplified ADAR1p150 aRNA Antisense Sequence
gaauacacgaauagagcaauu

SEQ ID NO: 54 - RNA, 21 nt, Artificial Sequence
  Exemplified ADAR1p150 aRNA Antisense Sequence
cuccaugcuucucgaguuauu

SEQ ID NO: 55 - RNA, 21 nt, Artificial Sequence
  Exemplified ADAR1p150 aRNA Antisense Sequence
agucuucagcgcuucucuauu

SEQ ID NO: 56 - RNA, 21 nt, Artificial Sequence
  Exemplified ADAR1p150 aRNA Antisense Sequence
ggaauacacgaauagagcauu

SEQ ID NO: 57 - RNA, 21 nt, Artificial Sequence
  Exemplified ADAR1p150 aRNA Antisense Sequence
gucugaaggcuuuaguaaauu

SEQ ID NO: 58 - RNA, 21 nt, Artificial Sequence
  Exemplified ADAR1p150 aRNA Antisense Sequence
ccuacuccaugcuucucgauu

SEQ ID NO: 59 - RNA, 21 nt, Artificial Sequence
  Exemplified ADAR1p150 aRNA Antisense Sequence
acggcuucccgugaaucaauu

SEQ ID NO: 60 - RNA, 21 nt, Artificial Sequence
  Exemplified ADAR1p150 aRNA Antisense Sequence
ccuacuauguuauaaacuuuu

SEQ ID NO: 61 - RNA, 21 nt, Artificial Sequence
  Exemplified ADAR1p150 aRNA Antisense Sequence
aauuacaaggcagucccuuuu

SEQ ID NO: 62 - RNA, 21 nt, Artificial Sequence
  Exemplified ADAR1p150 aRNA Antisense Sequence
uaaugcuccuacuauguuauu

SEQ ID NO: 63 - RNA, 21 nt, Artificial Sequence
  Exemplified ADAR1p150 aRNA Antisense Sequence
acacucauucaaauuacacuu

SEQ ID NO: 64 - RNA, 21 nt, Artificial Sequence
  Exemplified ADAR1p150 aRNA Antisense Sequence
gucuucagcgcuucucuacuu

SEQ ID NO: 65 - RNA, 21 nt, Artificial Sequence
  Exemplified ADAR1p150 aRNA Sense Sequence
agcauggaguaggaaaccauu

SEQ ID NO: 66 - RNA, 21 nt, Artificial Sequence
  Exemplified ADAR1p150 aRNA Antisense Sequence
ugguuuccuacuccaugcuuu

SEQ ID NO: 67 - RNA, 21 nt, Artificial Sequence
  Exemplified ADAR1p150 aRNA Sense Sequence
agcauggaguaggaaaccguu

SEQ ID NO: 68 - RNA, 21 nt, Artificial Sequence
  Exemplified ADAR1p150 aRNA Antisense Sequence
cgguuuccuacuccaugcuuu

SEQ ID NO: 69 - RNA, 21 nt, Artificial Sequence
  Exemplified ADAR1p150 aRNA Sense Sequence
agcauggaguaggaaacuauu

SEQ ID NO: 70 - RNA, 21 nt, Artificial Sequence
  Exemplified ADAR1p150 aRNA Antisense Sequence
uaguuuccuacuccaugcuuu

SEQ ID NO: 71 - RNA, 21 nt, Artificial Sequence
  Exemplified ADAR1p150 aRNA Sense Sequence
agcauggagcaggaaaccauu

SEQ ID NO: 72 - RNA, 21 nt, Artificial Sequence
  Exemplified ADAR1p150 aRNA Antisense Sequence
ugguuuccuguuccaugcuuu

SEQ ID NO: 73 - RNA, 21 nt, Artificial Sequence
  Exemplified ADAR1p150 aRNA Sense Sequence
agcauggaauaggaaaccauu

SEQ ID NO: 74 - RNA, 21 nt, Artificial Sequence
  Exemplified ADAR1p150 aRNA Antisense Sequence
ugguuuccuauuccaugcuuu

SEQ ID NO: 75 - RNA, 21 nt, Artificial Sequence
  Exemplified ADAR1p150 aRNA Sense Sequence
aacauggaguaggaaaccauu

SEQ ID NO: 76 - RNA, 21 nt, Artificial Sequence
  Exemplified ADAR1p150 aRNA Antisense Sequence
ugguuuccuacuccauguuuu

SEQ ID NO: 77 - RNA, 21 nt, Artificial Sequence
  Exemplified ADAR1p150 aRNA Sense Sequence
ggcauggaguaggaaaccauu

SEQ ID NO: 78 - RNA, 21 nt, Artificial Sequence
  Exemplified ADAR1p150 aRNA Antisense Sequence
ugguuuccuacuccaugccuu

SEQ ID NO: 79 - RNA, 21 nt, Artificial Sequence
  Exemplified ADAR1p150 aRNA Sense Sequence
gcuauaaagggacugccuuuu

SEQ ID NO: 80 - RNA, 21 nt, Artificial Sequence
  Exemplified ADAR1p150 aRNA Sense Sequence
gcuauaaagggacugccucuu

SEQ ID NO: 81 - RNA, 21 nt, Artificial Sequence
  Exemplified ADAR1p150 aRNA Antisense Sequence
gaggcagucccuuuauagcuu

SEQ ID NO: 82 - RNA, 21 nt, Artificial Sequence
  Exemplified ADAR1p150 aRNA Sense Sequence
gcuauaaagggacugcuuuuu

SEQ ID NO: 83 - RNA, 21 nt, Artificial Sequence
  Exemplified ADAR1p150 aRNA Antisense Sequence
aaagcagucccuuuauagcuu

SEQ ID NO: 84 - RNA, 21 nt, Artificial Sequence
  Exemplified ADAR1p150 aRNA Sense Sequence
gcuauaaagagacugccuuuu

SEQ ID NO: 85 - RNA, 21 nt, Artificial Sequence
  Exemplified ADAR1p150 aRNA Antisense Sequence
aaggcagucucuuuauagcuu

SEQ ID NO: 86 - RNA, 21 nt, Artificial Sequence
  Exemplified ADAR1p150 aRNA Sense Sequence
gcuauaaggggacugccuuuu

SEQ ID NO: 87 - RNA, 21 nt, Artificial Sequence
  Exemplified ADAR1p150 aRNA Antisense Sequence
aaggcaguccccuuauagcuu

SEQ ID NO: 88 - RNA, 21 nt, Artificial Sequence
  Exemplified ADAR1p150 aRNA Sense Sequence
gccauaaagggacugccuuuu

SEQ ID NO: 89 - RNA, 21 nt, Artificial Sequence
  Exemplified ADAR1p150 aRNA Antisense Sequence
aaggcagucccuuuauggcuu

SEQ ID NO: 90 - RNA, 21 nt, Artificial Sequence
  Exemplified ADAR1p150 aRNA Sense Sequence
acuauaaagggacugccuuuu

SEQ ID NO: 91 - RNA, 21 nt, Artificial Sequence
  Exemplified ADAR1p150 aRNA Antisense Sequence
aaggcagucccuuuauaguuu

SEQ ID NO: 92 - RNA, 21 nt, Artificial Sequence
  Exemplified ADAR1p150 aRNA Sense Sequence
cugcuauaaagggacugccuu

SEQ ID NO: 93 - RNA, 23 nt, Artificial Sequence
  Exemplified ADAR1p150 aRNA Antisense Sequence
aaggcagucccuuuauagcagua

SEQ ID NO: 94 - RNA, 21 nt, Artificial Sequence
  Exemplified ADAR1p150 aRNA Sense Sequence
uggcaucugcuugcuuaaguu

SEQ ID NO: 95 - RNA, 23 nt, Artificial Sequence
  Exemplified ADAR1p150 aRNA Antisense Sequence
aacuuaagcaagcagaugccagc

SEQ ID NO: 96 - RNA, 21 nt, Artificial Sequence
  Exemplified ADAR1p150 aRNA Sense Sequence
gaagcauggaguaggaaacca

SEQ ID NO: 97 - RNA, 23 nt, Artificial Sequence
  Exemplified ADAR1p150 aRNA Antisense Sequence
ugguuuccuacuccaugcuucuc

SEQ ID NO: 98 - RNA, 21 nt, Artificial Sequence
  Exemplified ADAR1p150 aRNA Sense Sequence
aguaaugguguaauuugaaug

SEQ ID NO: 99 - RNA, 23 nt, Artificial Sequence
  Exemplified ADAR1p150 aRNA Antisense Sequence
cauucaaauuacaccauuacuuu

SEQ ID NO: 100 - RNA, 21 nt, Artificial Sequence
  Exemplified ADAR1p110 aRNA Antisense Sequence
aaaccagcaaugcugcuuguu

SEQ ID NO: 101 - RNA, 21 nt, Artificial Sequence
  Exemplified ADAR1p110 aRNA Antisense Sequence
ucaaccuuucaaauuuacauu

SEQ ID NO: 102 - RNA, 21 nt, Artificial Sequence
  Exemplified ADAR1p110 aRNA Antisense Sequence
aaucaaccuuucaaauuuauu

SEQ ID NO: 103 - RNA, 21 nt, Artificial Sequence
  Exemplified ADAR1p110 aRNA Antisense Sequence
aggaucuaggucauaaaaauu

SEQ ID NO: 104 - RNA, 21 nt, Artificial Sequence
  Exemplified ADAR1p110 aRNA Antisense Sequence
caagguaucugggaaacgauu

SEQ ID NO: 105 - RNA, 21 nt, Artificial Sequence
  Exemplified ADAR1p110 aRNA Antisense Sequence
gcuccuacuauguuauaaauu

SEQ ID NO: 106 - RNA, 21 nt, Artificial Sequence
  Exemplified ADAR1p110 aRNA Antisense Sequence
gcaacagcagcagugaacauu

SEQ ID NO: 107 - RNA, 21 nt, Artificial Sequence
  Exemplified ADAR1p110 aRNA Antisense Sequence
aagauaacuuuuguuaucuuu

SEQ ID NO: 108 - RNA, 21 nt, Artificial Sequence
  Exemplified ADAR1p150 aRNA Antisense Sequence
aacuuaagcaagcagaugcuu

SEQ ID NO: 109 - RNA, 21 nt, Artificial Sequence
  Exemplified ADAR1p150 aRNA Antisense Sequence
gcuucucuacacucauucauu

SEQ ID NO: 110 - RNA, 21 nt, Artificial Sequence
  Exemplified ADAR1p150 aRNA Antisense Sequence
agaucucgucaaacgaccauu

SEQ ID NO: 111 - RNA, 21 nt, Artificial Sequence
  Exemplified ADAR1p150 aRNA Antisense Sequence
ccaugcuucucgaguuacuuu

SEQ ID NO: 112 - RNA, 21 nt, Artificial Sequence
  Exemplified ADAR1p150 aRNA Antisense Sequence
acugagcuccuaauuccccuu

SEQ ID NO: 113 - RNA, 21 nt, Artificial Sequence
  Exemplified ADAR1p150 aRNA Antisense Sequence
cauucaaauuacaccauuauu

SEQ ID NO: 114 - RNA, 21 nt, Artificial Sequence
  Exemplified ADAR2 aRNA Antisense Sequence
aaguggggaguaagagaccuu

SEQ ID NO: 115 - RNA, 21 nt, Artificial Sequence
  Exemplified ADAR2 aRNA Antisense Sequence
aucgcaagccagggauggguu

SEQ ID NO: 116 - RNA, 21 nt, Artificial Sequence
  Exemplified ADAR2 aRNA Antisense Sequence
accugggugggggaagcgguu

SEQ ID NO: 117 - RNA, 21 nt, Artificial Sequence
  Exemplified ADAR2 aRNA Antisense Sequence
acucugcccucggccucuuuu

SEQ ID NO: 118 - RNA, 21 nt, Artificial Sequence
  Exemplified ADAR2 aRNA Antisense Sequence
acuagcccuggccuccaaauu

SEQ ID NO: 119 - RNA, 21 nt, Artificial Sequence
  Exemplified ADAR2 aRNA Antisense Sequence
augggguggcgcacacuguuu

SEQ ID NO: 120 - RNA, 21 nt, Artificial Sequence
  Exemplified ADAR2 aRNA Antisense Sequence
cacucugcccucggccucuuu

SEQ ID NO: 121 - RNA, 21 nt, Artificial Sequence
  Exemplified ADAR2 aRNA Antisense Sequence
cagguguggacaagcggguuu

SEQ ID NO: 122 - RNA, 21 nt, Artificial Sequence
  Exemplified ADAR2 aRNA Antisense Sequence
cccucggccucuuuccuccuu

SEQ ID NO: 123 - RNA, 21 nt, Artificial Sequence
  Exemplified ADAR2 aRNA Antisense Sequence
ccuggccuccaaaccucaguu

SEQ ID NO: 124 - RNA, 21 nt, Artificial Sequence
  Exemplified ADAR2 aRNA Antisense Sequence
cucggccucuuuccucccuuu

SEQ ID NO: 125 - RNA, 21 nt, Artificial Sequence
  Exemplified ADAR2 aRNA Antisense Sequence
cuggccuccaaaccucagguu

SEQ ID NO: 126 - RNA, 21 nt, Artificial Sequence
  Exemplified ADAR2 aRNA Antisense Sequence
ggccuccaaaccucaggcguu

SEQ ID NO: 127 - RNA, 21 nt, Artificial Sequence
  Exemplified ADAR2 aRNA Antisense Sequence
ggugggcagagccagguguuu

SEQ ID NO: 128 - RNA, 21 nt, Artificial Sequence
  Exemplified ADAR2 aRNA Antisense Sequence
gguguggacaagcgggugguu

SEQ ID NO: 129 - RNA, 21 nt, Artificial Sequence
  Exemplified ADAR2 aRNA Antisense Sequence
guaaaguggggaguaagaguu

SEQ ID NO: 130 - RNA, 21 nt, Artificial Sequence
  Exemplified ADAR2 aRNA Antisense Sequence
guacuagcccuggccuccauu

SEQ ID NO: 131 - RNA, 21 nt, Artificial Sequence
  Exemplified ADAR2 aRNA Antisense Sequence
guggacaagcgggugggucuu

SEQ ID NO: 132 - RNA, 21 nt, Artificial Sequence
  Exemplified ADAR2 aRNA Antisense Sequence
guggggaguaagagaccauuu

SEQ ID NO: 133 - RNA, 21 nt, Artificial Sequence
  Exemplified ADAR2 aRNA Antisense Sequence
gugggggaagcggcagguuuu

SEQ ID NO: 134 - RNA, 21 nt, Artificial Sequence
  Exemplified ADAR1p150 aRNA Sense Sequence
gcaucugcuugcuuaaguuuu

SEQ ID NO: 135 - RNA, 21 nt, Artificial Sequence
  Non-targeting sense sequence
uccuaugacuguagauuuuau

SEQ ID NO: 136 - RNA, 23 nt, Artificial Sequence
  Non-targeting antisense sequence
auaaaaucuacagucauaggaau

(19) United States
(12) Patent Application Publication
(10) Pub. No.: US 2024/0392297 A1
(43) Pub. Date: Nov. 28, 2024

(54) RNA THERAPEUTICS AND METHODS OF USE THEREOF

(71) Applicant: Eli Lilly and Company, Indianapolis, IN (US)

(72) Inventors: Ellese CARMONA, Boston, MA (US); Zhefeng LI, Cambridge, MA (US); Christalyn Sims RHODES, Carmel, IN (US)

(21) Appl. No.: 18/258,698

(22) PCT Filed: Dec. 20, 2021
(86) PCT No.: PCT/US2021/064367

§ 371 Date: Jun. 21, 2023

Related U.S. Application Data
(60) Provisional application No. 63/285,311, filed on Dec. 2, 2021.
(60) Provisional application No. 63/203,303, filed on Jul. 16, 2021.
(60) Provisional application No. 63/151,093, filed on Feb. 19, 2021.
(60) Provisional application No. 63/129,878, filed on Dec. 23, 2020.

(51) Int. Cl. C12N 15/113; C12N 9/78

(52) U.S. Cl. CPC C12N 15/1137; C12N 9/78; C12Y 305/04; C12N 2310/11

(57) ABSTRACT

ADAR activating RNA, RNA therapeutics comprising an ADAR activating RNA and methods of using same.

The present invention is in the field of medicine. More particularly, the present invention relates to RNA editing therapeutics.

RNA editing, in nature, is a biological process by which RNA is post-transcriptionally modified. Similar to DNA editing, RNA editing is an enzymatically catalyzed process which makes discrete changes to nucleotides. However, unlike DNA editing, RNA editing alters nucleotides at the RNA level (e.g., messenger RNA (mRNA), double stranded RNA (dsRNA), micro RNA (miRNA), and the like) but does not alter the genome.

Adenosine deaminase acting on RNA enzyme (ADAR) is a protein family involved in RNA editing. ADAR can make specific base changes to double stranded RNA (dsRNA) during transcription, changing adenosines of dsRNA to inosines in the resulting mRNA. In humans, four isoforms of ADAR (ADAR1p110, ADAR1p150, ADAR2 and ADAR3) are known. ADAR1p110, ADAR1p150 and ADAR2 are known to be involved in the adenosine-to-inosine editing of RNA, which plays a role in innate immunity and RNA editing of nervous system tissue, among other functions. ADAR3 is believed to negatively regulate RNA editing through competing with other ADAR proteins (e.g., ADAR 1 and 2) for binding to target transcripts. Overall, ADAR-catalyzed adenosine-to-inosine RNA editing is understood to be protective and contribute to expanding function and diversity of transcripts. Dysregulation of ADAR-catalyzed RNA editing, however, has been associated with several disorders including autoimmune and neurodegenerative disorders.

The concept of utilizing RNA as a therapeutic, whereby translation of mRNA is increased or decreased, is in its infancy and continuing to expand. Examples of such RNA therapeutics include RNA interference, RNA activation, and guide RNA-directed target RNA editing using systems such as CRISPR-Cas systems. Although promising, the use of RNA editing therapeutics to treat human disease remains in its infancy, facing numerous obstacles. For example, RNA editing therapeutics, which employ ADAR-catalyzed adenosine-to-inosine base editing, have proven to have efficacy issues due to poor ADAR activity. ADAR expression in adult human tissue is known to be lower than other organisms and also varies based on tissue system. As a result, ADAR catalyzed RNA editing systems are regarded as less efficient. To overcome these known problems, synthetically produced ADAR, including modified ADAR, have been developed. However, utilization of synthetic ADAR with RNA editing therapies has proven challenging, presenting issues related to significant off-target editing, immunogenicity, toxicity, and delivery. Thus, there remains a need for RNA editing therapies, which employ ADAR-catalyzed editing, for use in the treatment of human disease, that overcomes one or more of these challenges.

Accordingly, the present disclosure provides compositions and methods which address one of more of the challenges outlined above. More particularly, embodiments of the present disclosure provide an adenosine deaminase acting on RNA enzyme (“ADAR”) activating RNA (aRNA) which upregulates expression of ADAR. According to embodiments of the present disclosure, ADAR is ADAR1p110, ADAR1p150, ADAR2 or ADAR3. In some embodiments, the ADAR aRNA is approximately 15 to approximately 50 nucleotides, and in even further embodiments, the ADAR aRNA is approximately 19 to approximately 30 nucleotides. In some more specific embodiments, the ADAR aRNA is 21 or 22 nucleotides.

According to embodiments, ADAR aRNA comprises a sense and an antisense sequence. The ADAR aRNA antisense sequence is complementary to an ADAR target genomic sequence, with the sense strand sequence being complementary to the ADAR target sequence complement strand sequence. In some such embodiments, the ADAR aRNA sense and antisense are at least 80% complementary to their respective target sequence. According to embodiments of the present disclosure, the ADAR aRNA target sequence is within−3000 to +150 nucleotides of the ADAR target sequence transcription start site. According to some embodiments, the ADAR aRNA target sequence is within SEQ ID NOs: 1, 2, 3, 4, 5, 6, 7, 8, 9 10, 11, 12 and/or 13. According to specific embodiments of ADAR aRNA provided herein, the ADAR aRNA is an ADAR1p110 aRNA given by one or more of SEQ ID NOs. 14-36 and 100-107. According to specific embodiments of ADAR aRNA provided herein, the ADAR aRNA is an ADAR1p150 aRNA given by one or more of SEQ ID NOs. 37-99, 108-113 and 134. According to specific embodiments of ADAR aRNA provided herein, the ADAR aRNA is an ADAR2 aRNA given by one or more of SEQ ID NOs 114-133. According to specific embodiments of ADAR aRNA provided herein, the ADAR aRNA is an ADAR3 aRNA. According to some such embodiments, the target sequence is within SEQ ID NOs: 12 and/or 13.

In some embodiments of the present disclosure, the ADAR aRNA comprises a 3′ tail on one of, or both of, the sense and antisense strands. In some embodiments, the ADAR aRNA comprises at least one modified nucleotide. In some such embodiments, the at least one modified nucleotide comprises a nucleotide modification from at least one of a thio-modified, an amino-modified, a phosphate-modified, a cholesterol-triethylene glycol (TEG)-modified, a methyl-modified, and a fluoro-modified nucleotide.

According to some embodiments of the present disclosure, the ADAR aRNA comprises a single strand. According to other embodiments of the present disclosure, the ADAR aRNA comprises a duplex having an antisense strand and a sense strand. In some such embodiments, the antisense strand and the sense strand are each independently approximately 15 to approximately 50 nucleotides and in even further embodiments, the antisense strand and the sense strand are each independently approximately 19 to approximately 30 nucleotides. In even further embodiments, the antisense strand and the sense strand are each independently approximately 21 nucleotides. In some embodiments, at least one of the antisense strand and the sense strand independently comprise a 3′ overhang.

In some embodiments of the present disclosure, the ADAR aRNA provided herein is encoded on a nucleic acid vector.

Additionally, according to some embodiments of the ADAR aRNA provided herein, the aRNA is linked to a ligand targeting moiety. In some such embodiments the ligand targeting moiety is GalNAc.

According to some further embodiments, the ADAR aRNA provided herein is linked to a second RNA. According to some such embodiments, the second RNA is a therapeutic RNA. In some embodiments, the therapeutic RNA comprises one of an antisense oligonucleotide (ASO), including interfering RNA (iRNA) and micro RNA (miRNA); messenger RNA (mRNA); guide RNA (gRNA) including single guide RNA (sgRNA); or activating RNA (aRNA).

According to embodiments of the ADAR aRNA provided herein, the ADAR aRNA binds Argonaute-2 protein (AGO2) protein.

According to some embodiments, the ADAR aRNA is linked to a delivery vehicle. In some embodiments, the delivery vehicle comprises at least one of an antibody, or fragment thereof, a scFv, a peptide, GalNAc, an apatamer or a nanoparticle.

According to some further embodiments, the ADAR aRNA provided herein is encapsulated, fully or partially, within a delivery vehicle. According to some such embodiments, the delivery vehicle comprises at least one of a lipid, liposome, lipoplex, polymer or nanoparticle.

Additionally, methods of modulating expression of ADAR are provided herein, whereby a patient is administered an ADAR aRNA of the present disclosure. According to some such methods, ADAR expression is increased. In some such methods, ADAR expression is increased by at least 20%, by at least 30%, by at least 40%, by at least 50%, by at least 100%, by at least 150%, by at least 200%, by at least 250%, by at least 300%, or by at least 350%.

According to some embodiments of the present disclosure, a method of treating disease in a human is provided comprising administering a therapeutically effective amount of an ADAR aRNA of the present disclosure. In a particular embodiment, the ADAR aRNA is an ADAR3 aRNA, wherein the ADAR3 target sequences is within SEQ ID NO: 12 and/or 13. In some such embodiments, the ADAR3 aRNA is delivered to tissue exhibiting overexpression of ADAR1 and/or ADAR2. According to some embodiments, the disease is characterized by ADAR1-catalyzed or ADAR2-catalyzed hyperactive transcript editing. According to some embodiments, the disease is one of cancer, tumorigenesis, metastasis, brain cancer, a chronic neurological disorder, an immune disease or an autoimmune disease. According to some embodiments, the ADAR3 aRNA is delivered to the CNS. In some embodiments, a therapeutically effective amount of a therapeutic RNA to the human is also administered to the human.

The present disclosure also provides RNA editing therapeutics comprising a therapeutic RNA and an ADAR aRNA as provided herein. According to some such embodiments, the therapeutic RNA comprises one of an mRNA, miRNA, sgRNA, aRNA, iRNA or ASO.

Further, methods of treating a disease in a human is provided by the present disclosure. According to embodiments, such methods comprise administering a therapeutically effective amount of an RNA editing therapeutic to the human, wherein the RNA therapeutic comprises a therapeutic RNA and an ADAR aRNA of the present disclosure. According to some embodiments, the therapeutic RNA and the ADAR aRNA are co-administered. In some embodiments, the therapeutic RNA and the ADAR aRNA are co-formulated. In even further embodiments, the therapeutic RNA and the ADAR aRNA are linked. In embodiments of such methods, at least one of the therapeutic RNA and the ADAR aRNA are encapsulated, fully or partially, within a delivery vehicle. According to some such embodiments, the delivery vehicle comprises one of a lipidoid, liposome, lipoplex, polymer or nanoparticle. In some embodiments, at least one of the therapeutic RNA and the ADAR aRNA are linked to a delivery vehicle. According to some such embodiments the delivery vehicle comprises one of an antibody, or fragment thereof, a scFv, a peptide, GalNAc, an apatamer or a nanoparticle.

Additionally, according to some embodiments, a pharmaceutical composition comprising a therapeutic RNA, an ADAR aRNA as provided herein and at least one pharmaceutically acceptable excipient is provided by the present disclosure. According to some embodiments, the therapeutic RNA comprises one of an mRNA, miRNA, sgRNA, aRNA, iRNA, or ASO.

Additionally, the present disclosure also provides methods of treating a disease in a human comprising administering to the human a therapeutically effective amount of a pharmaceutical composition of the present disclosure. According to such embodiments, the pharmaceutical composition comprises a therapeutic RNA, an ADAR aRNA as provided by the present disclosure, and a pharmaceutically acceptable excipient. According to some such embodiments, the therapeutic RNA and the ADAR aRNA are co-administered. In some embodiments, the therapeutic RNA and the ADAR aRNA are co-formulated. In some embodiments, the therapeutic RNA and the ADAR aRNA are linked. Further, in some embodiments, at least one of the therapeutic RNA and the ADAR aRNA are encapsulated, fully or partially, within a delivery vehicle. According to some such embodiments, the delivery vehicle comprises one of a lipid, liposome, lipoplex, polymer or nanoparticle. In some embodiments, at least one of the therapeutic RNA and the ADAR aRNA are linked to a delivery vehicle. According to some such embodiments, the delivery vehicle comprises one of an antibody, or fragment thereof, a scFv, a peptide, GalNAc, an apatamer or a nanoparticle.

As used herein, RNA editing therapeutics refer to systems or methods which employ therapeutic RNA for modulating, i.e., upregulating or downregulating, the translation of protein from RNA. Examples of RNA editing therapeutics include, but are not limited to, RNA interference, RNA activation, and guide RNA directed target RNA editing using systems such as CRISPR-Cas systems. Such RNA editing therapeutics employ therapeutic RNAs such as antisense oligonucleotides, including interfering RNA (iRNA, aka siRNA) and micro RNA (miRNA), mRNA, RNA aptamers, activating RNA (aRNA, aka saRNA) and guide RNA (gRNA) including single guide RNA (sgRNA). The term therapeutic RNA refers to the RNA oligonucleotide (e.g., ASO, iRNA, miRNA, mRNA, RNA aptamer, aRNA, gRNA, sgRNA and the like) utilized in an RNA editing therapeutic.

As used herein, ADAR activating RNA (ADAR aRNA) refers to RNA that modulates expression of endogenous ADAR. ADAR aRNA comprises an antisense sequence which is complementary, at least 80% complementary, to an ADAR aRNA target sequence. According to embodiments, the ADAR aRNA target sequence is located between −3000 (e.g., 3000 nucleotides upstream) and +150 (150 nucleotides down-stream) nucleotides of the ADAR target sequence transcription start site. According to more specific embodiments, the ADAR aRNA target sequence is located within SEQ ID NOs. 1, 2, 3, 4, 5, 6, 7, 8, 9, 10, 11, 12 and/or 13. In particular embodiments, for example, ADAR1 p110 aRNA target sequence may be located within SEQ ID NOs. 1, 2 and/or 3, whereas ADAR1 p150 aRNA target sequence may be located within SEQ ID NOs. 4, 5, 6 and/or 7, whereas ADAR2 aRNA target sequence may be located within SEQ ID NOs. 8, 9, 10 and/or 11, and whereas ADAR3 aRNA target sequence may be located within SEQ ID NOs. 12 and/or 13.

Additionally, according to the present disclosure, embodiments of the therapeutic RNA and ADAR aRNA may be chemically synthesized or recombinantly produced using methods known in the art. Furthermore, embodiments of the therapeutic RNA and/or ADAR aRNA may comprise one or more modified nucleotides as known in the art, including thio-modified, amino-modified, phosphate-modified, cholesterol-TEG-modified, methyl-modified, fluoro-modified nucleotides, and the like.

As used herein, “RNA” refers to ribonucleic acid, and ribonucleotide, interchangeably. RNA refers to both naturally and non-naturally occurring (artificial, synthetic), modified or unmodified nucleotides or polynucleotides.

As used herein, “linked” refers to two or more moieties physically associated or connected with one another, either directly or via one or more additional moieties that serves as a linking agent, via covalent or non-covalent bonding, to form a structure.

Argonaute-2 protein (AGO2), referred to herein, is a naturally occurring protein in humans, known for binding RNA. AGO2 is known to be involved in RNA interference and possess endonuclease activity.

As referred to herein, a delivery vehicle connotates a molecule utilized in conjunction with an ADAR aRNA or therapeutic RNA for delivering the RNA to the patient. Examples include an antibody, or fragment thereof, a scFv, a peptide, GalNAc, an apatamer, or a nanoparticle linked to the RNA. Other examples include a lipidoid, liposome, lipoplex, polymer, or nanoparticle in which the RNA is encapsulated, either fully or partially.

Pharmaceutical composition, as used herein, refers to a composition comprising an ADAR aRNA of the present disclosure, formulated into a dosage form such as a topical, intranasal, intratracheal, or injectable (e.g., intravenous, intraocular, intravitreal, intramuscular, intracardiac, intraperitoneal, subcutaneous), including liquid dosage forms, injectable preparations, pulmonary forms, and solid dosage forms. A pharmaceutical composition may also include at least one pharmaceutically acceptable excipient and may also include an ADAR aRNA formulated in a dosage form in conjunction with a therapeutic RNA.

As used interchangeably herein, “treatment” and/or “treating” and/or “treat” are intended to refer to all processes wherein there may be a total elimination, slowing or delaying, reduction in severity or frequency (e.g., episodes), interruption or stopping of the progression of disease and/or symptoms thereof, but does not require a total elimination of all disease symptoms. Treatment includes administration of an RNA editing therapeutic according to the present disclosure which includes administration of ADAR aRNA, to a human that would benefit from at least one of the above-listed processes, including: (a) inhibiting or slowing further progression of disease symptoms and effects, i.e., arresting its development or progression; (b) relieving the disease, i.e., causing an elimination or regression of disease, disease symptoms or complications thereof, and (c) preventing or reducing the frequency of disease episodes.

As may be used herein, the terms “about” or “approximately”, when used in reference to a particular recited numerical value or range of values, means that the value may vary from the recited value by no more than 10% (e.g., +/−10%). For example, as used herein, the expression “about 100” includes 90 and 110 and all values in between (e.g., 91, 92, 93, 94, etc.).

### EXAMPLES

### Example 1: Exemplary ADAR aRNA

Exemplary ADAR aRNA may be prepared substantially as described below. For each human ADAR isoform, an ADAR aRNA library may be generated and screened. Nucleotide sequence from about −3000 (3000 nucleotides upstream) to about +150 (150 nucleotides downstream) of the ADAR transcriptional start sequence (e.g., position 0) may be selected. More particularly, for ADAR1p110, target sequence regions within SEQ ID NOs. 1, 2 and/or 3 (e.g., ADAR1 p110 Targeting Regions A, B and C) may be selected. For ADAR1p150, target sequence regions within SEQ ID NOs. 4, 5, 6 and/or 7 (e.g., ADAR1 p150 Targeting Regions A, B, C and D) may be selected. Similarly, for ADAR2, target sequence regions within SEQ ID NOs. 8, 9, 10 and/or 11 (e.g., ADAR2 Targeting Regions A, B, C and D) may be selected and, for ADAR3, target sequence regions within SEQ ID NOs. 12 and/or 13 (e.g., ADAR3 Targeting Regions A and B) may be selected. Regions comprising repeated elements and CpG island sequences may then be screened out.

Thereafter, an initial library of nucleotide sequences from 15-50 nucleotides may be selected. In exemplary embodiments, sequences of 21 and 22 nucleotides may be chosen. Exemplified libraries of ADAR1_p110 aRNA antisense sequences (Table 4), ADAR1_p150 aRNA antisense sequences (Tables 1, 2, 6 and 9) and ADAR2 aRNA antisense sequences (Table 8) are provided herein.

Further screening may be done. Library candidates may be cross compared against the ADAR transcript region and miRNA library, whereby ADAR aRNA candidates overlapping therewith may be screened out. Also, any candidate ADAR aRNA that represent complementary matches may have the complement screened out (e.g., only one candidate of a complimentary pair would be selected). Additionally, ADAR aRNA candidates identified as cross-reactive with other genes (e.g., that are identical or within 1 base mismatch of another gene transcriptome) may also be screened out. Further screening may be undertaken, whereby ADAR aRNA candidates that target low expression exon regions can be screened out. From the ADAR aRNA candidate library, AGO2 binding prediction scores can also be used to rank candidates for progressing into in vitro and in vivo assessment.

ADAR aRNA (both the sense and antisense strands) may also include chemical modifications. Exemplary chemical modifications according to embodiments of the present disclosure include 2′-O-methyl (mG, mA, mC, or mU) and/or 2′-fluoro (fG, fA, fC, or fU) at the 2′-ribose location. Additionally, the phosphodiester backbone may be substituted with phosphonothioate in one or more nucleotides and a 5′-phosphorylation modification may be introduced, for example at the 5′ end of antisense strand. Furthermore, as noted herein, ADAR aRNA of the present disclosure may be linked to a delivery vehicle or ligand targeting moiety such as a cholesterol or GalNAc, for example linked to the ADAR aRNA via a triethylene glycol. Exemplary embodiments are provided in Table 1.

TABLE 1
Exemplary ADAR aRNA Sense and Antisense
Comprising Chemical Modifications
(ADAR p150 aRNA):

| | | SEQ |
|---|---|---|
| Exemp. | | ID |
| Embods. | | NO. |
| 1 | sense: 5′ | 92 |
| | mC*mU*mGmCmUmAfUmAfAfAfGmGmGmAmCmUm | |
| | GmCmC*mU*mU 3′ | |
| | antisense: 5′ Phos | 93 |
| | mA*fA*mGmGmCfAmGmUmCmCmCmUmUfUmAfUm | |
| | AmGmCmAmG*mU*mA 3′ | |
| 2 | sense: 5′ | 94 |
| | mU*mG*mGmCmAmUfCmUfGfCfUmUmGmCmUmUm | |
| | AmAmG*mU*mU 3′ | |
| | antisense: 5′ Phos | 95 |
| | mA*fA*mCmUmUfAmAmGmCmAmAmGmCfAmGfAm | |
| | UmGmCmCmA*mG*mC 3′ | |
| 3 | sense: 5′ | 96 |
| | mG*mA*mAmGmCmAfUmGfGfAfGmUmAmGmGmAm | |
| | AmAmC*mC*mA 3′ | |
| | antisense: 5′ Phos | 97 |
| | mU*fG*mGmUmUfUmCmCmUmAmCmUmCfCmAfUm | |
| | GmCmUmUmC*mU*mC 3′ | |
| 4 | sense: 5′ | 98 |
| | mA*mG*mUmAmAmUfGmGfUfGfUmAmAmUmUmUm | |
| | GmAmA*mU*mG 3′ | |
| | antisense: 5′ Phos | 99 |
| | mC*fA*mUmUmCfAmAmAmUmUmAmCmAfCmCfAm | |
| | UmUmAmCmU*mU*mU 3′ | |

(*= phosphonothioate);
(mG, mA, mC, or mU = 2′-O-methyl);
(fG, fA, fC, or fU = 2′-fluoro);
(Phos = phosphorylation)

Additionally, sense and antisense strands of ADAR aRNA of the present disclosure may include nucleotide overhangs of one, two, or even up to five nucleotides. According to some embodiments, one or both the sense and antisense strands may include one, two, or even up to five nucleotides at the 5′ ends. In some embodiments, one or both of the sense and antisense strands include one, two, or even up to five nucleotides at the 3′ ends. Some embodiments, both eh sense and antisense strands include a two uracil 3′ overhang.

Additionally, as noted herein, one or both of the sense and antisense strands of an ADAR aRNA provided herein, may include one or more nucleotide mis-matches between the nucleotide sequences of the target sequences for both the sense and antisense strands. Exemplified embodiments including mismatch nucleotide sequences of both the sense and antisense strands are provided in Table 2 (the sense and antisense sequences of Ref A and Ref. B in Table 2 do not include mismatches to their respective ADAR target sequences). Modulation of ADAR1 p150 expression by exemplary ADAR aRNA having mismatches is performed according to the process provided in Example 2B herein. Percent of ADAR expression levels as compared to Ref. A or Ref. B, respectively, is set forth in Table 2.

TABLE 2
Exemplary ADAR aRNA Sense and
Antisense w/ Mismatches:

| | | | | % |
|---|---|---|---|---|
| | Sequences (5′ → 3′, | | SEQ | Expression |
| Exemp. | with each strand having | | ID | compared |
| Embods. | a UU 3′ overhang) | | NO. | to Ref. |
| Ref. A | sense: | AGCAUGGAGUAGGAAACCAUU | 65 | 100% |
| (ADAR | anti- | UGGUUUCCUACUCCAUGCUUU | 66 | |
| p150 | sense: | | | |
| Targeting | | | | |
| Region D) | | | | |
| 1 | sense: | AGCAUGGAGUAGGAAACCGUU | 67 | 54.67% |
| | anti- | CGGUUUCCUACUCCAUGCUUU | 68 | |
| | sense: | | | |
| 2 | sense: | AGCAUGGAGUAGGAAACUAUU | 69 | 52.91% |
| | anti- | UAGUUUCCUACUCCAUGCUUU | 70 | |
| | sense: | | | |
| 3 | sense: | AGCAUGGAGCAGGAAACCAUU | 71 | 78.16% |
| | anti- | UGGUUUCCUGUUCCAUGCUUU | 72 | |
| | sense: | | | |
| 4 | sense: | AGCAUGGAAUAGGAAACCAUU | 73 | 88.34% |
| | anti- | UGGUUUCCUAUUCCAUGCUUU | 74 | |
| | sense: | | | |
| 5 | sense: | AACAUGGAGUAGGAAACCAUU | 75 | 101.29% |
| | anti- | UGGUUUCCUACUCCAUGUUUU | 76 | |
| | sense: | | | |
| 6 | sense: | GGCAUGGAGUAGGAAACCAUU | 77 | 118.03% |
| | anti- | UGGUUUCCUACUCCAUGCCUU | 78 | |
| | sense: | | | |
| Ref. B | sense: | GCUAUAAAGGGACUGCCUUUU | 79 | 100% |
| (ADAR | anti- | AAGGCAGUCCCUUUAUAGCUU | 37 | |
| p150 | sense: | | | |
| Targeting | | | | |
| Region B) | | | | |
| 1 | sense: | GCUAUAAAGGGACUGCCUCUU | 80 | 53.49% |
| | anti- | GAGGCAGUCCCUUUAUAGCUU | 81 | |
| | sense: | | | |
| 2 | sense: | GCUAUAAAGGGACUGCUUUUU | 82 | 142.62% |
| | anti- | AAAGCAGUCCCUUUAUAGCUU | 83 | |
| | sense: | | | |
| 3 | sense: | GCUAUAAAGAGACUGCCUUUU | 84 | 67.10% |
| | anti- | AAGGCAGUCUCUUUAUAGCUU | 85 | |
| | sense: | | | |
| 4 | sense: | GCUAUAAGGGGACUGCCUUUU | 86 | 88.73% |
| | anti- | AAGGCAGUCCCCUUAUAGCUU | 87 | |
| | sense: | | | |
| 5 | sense: | GCCAUAAAGGGACUGCCUUUU | 88 | 101.44% |
| | anti- | AAGGCAGUCCCUUUAUGGCUU | 89 | |
| | sense: | | | |
| 6 | sense: | ACUAUAAAGGGACUGCCUUUU | 90 | 71.59% |
| | anti- | AAGGCAGUCCCUUUAUAGUUU | 91 | |
| | sense: | | | |

### Example 2: In-Vitro ADAR Expression Modulation

Modulation of ADAR expression by ADAR aRNA candidates prepared according to the process provided herein may be assessed substantially as described herein. ADAR aRNA may be transfected into the target cell line at serial concentrations for 24 hours. Individual ADAR isoform expression, in mRNA and/or protein level, may then be assayed individually. ADAR mRNA levels are assayed using quantitative reverse-transcription polymerase chain reaction (qRT-PCR) whereas ADAR protein expression level is able to be assayed by western blot or enzyme-linked immunosorbent assay (ELISA). Expression levels of ADAR mRNA and protein may then be compared to untreated controls to identify ADAR aRNA candidates providing the greatest percent upregulation.

### Example 2A. ADAR1 p110

Expression levels of ADAR1 mRNA in HEK293T cells, transfected with ADAR1 p110 aRNA are assessed and compared to control ADAR1 mRNA levels of HEK293T cells not transfected with ADAR1 p110 aRNA, substantially as described herein. Briefly, HEK293T cells are seeded at 10,000 cells/well on a 96-well plate in serum-free medium (Opti-MEM, Catalog #11058021). Thereafter, cells are either treated with 100 nM of an ADAR p110 aRNA (antisense sequence as set forth in Table 4) with vehicles (Lipofectamine RNAiMax, Catalog #13778100) or with vehicles alone as controls (not transfected with an ADAR p110 aRNA) for no more than 12 hours. Media on treated and untreated cells are changed to full media (DMEM+10% FBS) 8-12 hours post-transfection to maintain cell culture until endpoint. ADAR1 mRNA levels are measured using quantitative RT-PCR (PowerTrack SYBR Green Master Mix, Catalog #A46012) according to manufacturer's instructions (SYBR Green Fast Advanced Cells-to-cT Kit, Catalog #A35379). As demonstrated in Table 4a, three specific ADAR1 p110 Targeting Regions (denoted Region A, B, and C in Table 3) are identified and ADAR1 p110 aRNA anti sense sequences targeting one of those three regions demonstrate an increase of up to 15000 ADAR1 transcription.

TABLE 3
ADAR1 p110 Targeting Regions:

| | |
|---|---|
| | Region −715 to −322 |
| Region A | (Reversed to 5′-3′) |

TTTGTTAAGATATATATATATTTTTTTTTTTTTAAGCACTCCTTTGAAAG
GATTAAGGACGCCTAACTTGAAGGAAAAGCATTTCTGCACAGGTGTCAGT
GTATTGCACTGTGGAACCTGTGTGGTAAAGGCAAAGGGGGTAGTGCTTAT
CTCTTGATCCTAAATATGTGAGACCAGATTAAAGTGAAATCTGGGAGGCA
ATGAATGTTAAATGAGTTGTTATGTAATTTGCATAGAGGTGATGCTGAGA
GATTTAGAAAGGATCACTGTGGGTTGCTTGCTCACTTTCTTGCTCTCCTA
TTCCGTAGCTTTCCAAATGGCTGTACTCAACGGTGGCTTGGTGTTTAGGG
GATTTAAGGGGGGCAAAAAGAAAGATTAATAATCTCCTCCTCTC (SEQ
ID NO: 1)

| | |
|---|---|
| | Region −1469 to −1137 |
| Region B | (Reversed to 5′-3′) |

AGTCTTGCCAAGCAGCATTGCTGGTTTAGGAATTTGTGAATTTGTATCCT
GCTCATTAATTCTGCAGAATGGAGCAGTGCGTGAAGAGGGCTTGGGGGAA
AATGCGCCCCCGTCTGAGTAGGAAGGCCTGAGCCCATGTCAAGGCAGACA
CATCGTCTCCCTTTCTGCTAGGGCCCCTTGTGGAACCCCCTACCCCCGCT
TTAGCCCCACTTGAACAACGTTCGGACTTTGAGCAGCGCACACTATCCTC
AGCTCACCTTATCCACCTCCTGAAGGCCTTCTGGGAGTTAAAAATGGCAC
TTAAGCTGTAGGAGAAAGCTTGTTAACCACTTT (SEQ ID NO: 2)

| | |
|---|---|
| | Region −1619 to −1500 |
| Region C | (Reversed to 5′-3′) |

TCGTCTTGCCAAGCAGCATTGCTGGTTTAGGAATTTGTGCGTCTTGTGAG
TGTGTGTGTGTGGGTGTGTGTCGTCTTGCCAAGCAGCATTGCTGGTTTAG
GAATTTGTGCGTCTTGTGAGA (SEQ ID NO: 3)

TABLE 4a
Exemplified ADAR1 p110 aRNA Antisense Sequences and % Increase in
ADAR1 Transcription Expression:

| | ADAR1_ | % Increase in |
|---|---|---|
| aRNA Antisense Sequence | p110 Region | ADAR1 expression |
| GCAAGACGACACACACCCAUU (SEQ ID NO: 14) | C | 141.73; S.D. (15.97) |
| UGCUUGGCAAGACGACACAUU (SEQ ID NO: 15) | C | 59.84; S.D. (8.61) |
| ACGGAAUAGGAGAGCAAGAUU (SEQ ID NO: 16) | A | 140.67; S.D. (15.50) |
| CAAGUUAGGCGUCCUUAAUUU (SEQ ID NO: 17) | A | 136.01; S.D. (18.96) |
| GCUUUCUCCUACAGCUUAAUU (SEQ ID NO: 18) | B | 159.87; S.D. (28.23) |
| CUUUCAAAGGAGUGCUUAAUU (SEQ ID NO: 19) | A | 135.87; S.D. (11.27) |
| AUGCUGCUUGGCAAGACGAUU (SEQ ID NO: 20) | C | 129.53; S.D. (18.52) |
| CUAGCAGAAAGGGAGACGAUU (SEQ ID NO: 21) | B | 142.67; S.D. (22.53) |
| AAAGUGAGCAAGCAACCCAUU (SEQ ID NO: 22) | A | 133.95; S.D. (10.78) |
| AGUCCGAACGUUGUUCAAGUU (SEQ ID NO: 23) | B | 154.88; S.D. (18.46) |
| AGGAUACAAAUUCACAAAUUU (SEQ ID NO: 24) | B | 135.32; S.D. (16.73) |
| AAGUUAGGCGUCCUUAAUCUU (SEQ ID NO: 25) | A | 135.53; S.D. (13.06) |
| CACCUGUGCAGAAAUGCUUUU (SEQ ID NO: 26) | A | 164.61; S.D. (22.29) |
| UUACCACACAGGUUCCACAUU (SEQ ID NO: 27) | A | 155.21; S.D. (23.49) |
| CAAGAGAUAAGCACUACCCUU (SEQ ID NO: 28) | A | 130.13; S.D. (19.71) |
| UAAAUCCCCUAAACACCAAUU (SEQ ID NO: 29) | A | 143.91; S.D. (29.04) |
| UUAAAUCCCCUAAACACCAUU (SEQ ID NO: 30) | A | 137.27; S.D. (17.39) |
| UUUCUCCUACAGCUUAAGUUU (SEQ ID NO: 31) | B | 178.39; S.D. (30.140) |
| ACUCAUUUAACAUUCAUUGUU (SEQ ID NO: 32) | A | 134.26; S.D. (8.02) |
| UCUCCUACAGCUUAAGUGCUU (SEQ ID NO: 33) | B | 138.19; S.D. (12.51) |
| CUUAAAUCCCCUAAACACCUU (SEQ ID NO: 34) | A | 151.05; S.D. (16.91) |
| UUCUCCUACAGCUUAAGUGUU (SEQ ID NO: 35) | B | 164.56; S.D. (12.68) |
| AUAAGCACUACCCCCUUUGUU (SEQ ID NO: 36) | A | 138.34; S.D. (26.39) |
| AAACCAGCAAUGCUGCUUGUU (SEQ ID NO: | B | 133.95; S.D. (10.78) |
| 100) | | |

Expression levels of ADAR1 P110 mRNA in HELA cells, transfected with ADAR1 p110 aRNA are assessed and compared to control ADAR1 P 110 levels of HELA cells not transfected with ADAR1 P110 aRNA, substantially as described herein. Briefly, HELA cells are seeded 10,000 cells/well on 96-well plate setting in serum-free medium (Opti-MEM, Catalog #11058021). Thereafter, cells are either treated with 100 nM of an ADAR p110 aRNA (antisense sequence as set forth in Table 4b) with vehicles (Lipofectamine RNAiMax, Catalog #13778100) or with vehicles alone as controls (not transfected with an ADAR p110 aRNA) for no more than 12 hours. Media on treated and untreated cells are changed to full media (DMEM+1000 FBS) 8-12 hours post-transfection to maintain cell culture until endpoint. ADAR1 P110 mRNA levels are measured using quantitative RT-PCR (TaqMan™ Fast Advanced Master Mix, Catalog #4444965) from cDNA synthesized according to manufacturer's instructions (SYBR Green Fast Advanced Cells-to-cT Kit, Catalog #A35379). As demonstrated in Table 4b, three specific ADAR1 P110 Targeting Regions (denoted Region A, B, and C in Table 3) are identified and ADAR1 P110 aRNA anti sense sequences targeting one of those three regions demonstrate an increase of up to 3500% ADAR1 P110 transcription.

TABLE 4b
Exemplified ADAR1 p110 aRNA Antisense Sequences and %
Increase in ADAR1 Transcription Expression:

| | ADAR1_p110 | % Increase in |
|---|---|---|
| aRNA Antisense Sequence | Region | ADAR1 expression |
| AAGGCAGUCCCUUUAUAGCUU | C | 351.5; S.D. (59.5) |
| (SEQ ID NO: 37) | | |
| UCCUACUCCAUGCUUCUCGUU | A | 308.7; S.D. (145.9) |
| (SEQ ID NO: 40) | | |
| UCAACCUUUCAAAUUUACAUU | A | 233.8; S.D. (65) |
| (SEQ ID NO: 101) | | |
| CUACUAUGUUAUAAACUUAUU | A | 281.2; S.D. (47.7) |
| (SEQ ID NO: 51) | | |
| AAUCAACCUUUCAAAUUUAUU | A | 254.7; S.D. (39.2) |
| (SEQ ID NO: 102) | | |
| AGGAUCUAGGUCAUAAAAAUU | A | 281.5; S.D. (63.3) |
| (SEQ ID NO: 103) | | |
| CAAGGUAUCUGGGAAACGAUU | A | 239.2; S.D. (57.1) |
| (SEQ ID NO: 104) | | |
| GCUCCUACUAUGUUAUAAAUU | A | 232.5; S.D. (46.9) |
| (SEQ ID NO: 105) | | |
| CCUACUAUGUUAUAAACUUUU | B | 256.6; S.D. (28.3) |
| (SEQ ID NO: 60) | | |
| GCAACAGCAGCAGUGAACAUU | A | 238.3; S.D. (111) |
| (SEQ ID NO: 106) | | |
| AAGAUAACUUUUGUUAUCUUU | A | 439.5; S.D. (166.4) |
| (SEQ ID NO: 107) | | |

### Example 2B. ADAR1 p150

Expression levels of ADAR1 p150 mRNA in HEK293T cells, transfected with ADAR1 p150 aRNA are assessed and compared to control ADAR1 mRNA levels of HEK293T cells not transfected with ADAR1 p150 aRNA, substantially as described herein. Briefly, HEK293T cells are seeded at 10,000 cells/well on 96-well plate in serum-free medium (Opti-MEM, Catalog #11058021). Thereafter, cells are either treated with 100 nM of an ADAR p150 aRNA (antisense sequence as set forth in Table 6a) with vehicles (Lipofectamine RNAiMax, Catalog #13778100) or by vehicles alone as controls (not transfected with an ADAR p150 aRNA) for no more than 12 hours. Media on treated and untreated cells are changed to full media (DMEM+10% FBS) 8-12 hours post-transfection to maintain cell culture until endpoint. ADAR1 p150 mRNA transcription are measured using quantitative RT-PCR (PowerTrack SYBR Green Master Mix, Catalog #A46012) according to manufacturer's instructions (SYBR Green Fast Advanced Cells-to-cT Kit, Catalog #A35379). As demonstrated in Table 6a, four specific ADAR1 p150 Targeting Regions (denoted Region A, B, C and D in Table 5) are identified and ADAR1 p150 aRNA antisense sequences targeting one of those four regions demonstrate an increase of up to 370% ADAR1 transcription.

TABLE 5
ADAR1 p150 Targeting Regions:

| | |
|---|---|
| | Region −828 to −456 |
| Region A | (Reversed to 5′-3′) |

TGGGGTAGTTTTTATGACCTAGATCCTAAATTGTTCACTGCTGCTGTTGC
TACTCTTGGTACTTTTTACTGGCTGGCATCTGCTTGCTTAAGTTTATAAC
ATAGTAGGAGCATTAACAAGGTCCCACGGTGGGGACCTTGGTCGTTTGAC
GAGATCTGCGCTCCCGCCCATCCCCTCCCCCCCCCCTCCACATTGGAGAC
GCGGCCACCACCGCGCTGGCGCGGAGAGAGGGAGGACCGGGCGTCATGCT
GTTTCTGGCCTGAGGTTTTGTGTGCCTTTGTTTTCCTTTTGCTCTATTCG
TGTATTCCTGCCTACGGCCTGTGCGGGGAATTAGGAGCTCAGTACTGAAA
CGGCGGTTTTCCTAAACAGTACC (SEQ ID NO: 4)

| | |
|---|---|
| | Region −1136 to −934 |
| Region B | (Reversed 5′-3′) |

AATCGTTTCCCAGATACCTTGAACAAAAATCCAGCAGTTAGAGAAGCCTG
ACCATGAAGCAAATTTGACTTTTGTCCCTCTAGATAACAAAAGTTATCTT
TTTGAAAGTAATGGTGTAATTTGAATGAGTGTAGAGAAGCGCTGAAGACT
GAGCTTTACTAAAGCCTTCAGACCTGGATTTGGCAGCAGCGTGGCCTTAG
TCA (SEQ ID NO: 5)

| | |
|---|---|
| | Region −1274 to −1211 |
| Region C | (Reversed 5′-3′) |

AACACCATGAAAGGGCATCAGCTGGAGATACTGCTATAAAGGGACTGCCT
TGTAATTTCATA (SEQ ID NO: 6)

| | |
|---|---|
| | Region −1539 to −1370 |
| Region D | Reversed 5′-3′) |

GGGGCGTTTTTAGCGCAGTGTGCAAGTGCCCTATTAGGGGTAGGCGCCCA
GTAACTCGAGAAGCATGGAGTAGGAAACCACAAACAGCACCTGCTCCCCC
TCCTCTCCCCCTACCTGCTGTGGGGAAGGCCTCCCTTGTAAATTTGAAAG
GTTGATTCACGGGAAGCCGT (SEQ ID NO: 7)

TABLE 6a
Exemplified ADARI p150 aRNA Antisense
Sequences and % Increase in ADAR1
Transcription Expression:

| RNA antisense | ADAR1_p150 | % Increase in |
|---|---|---|
| sequence | Region | ADAR1 expression |
| AAGGCAGUCCCUUUAUAGCUU | B | 370.11 (S.D. 49.07) |
| (SEQ ID NO: 37) | | |
| UAUAGCAGUAUCUCCAGCUUU | B | 241.89 (S.D. 30.89) |
| (SEQ ID NO: 38) | | |
| UUCAGCGCUUCUCUACACUUU | C | 302.78 (S.D. 28.17) |
| (SEQ ID NO: 39) | | |
| UCCUACUCCAUGCUUCUCGUU | D | 215.84 (S.D. 44.78) |
| (SEQ ID NO: 40) | | |
| UUUUUGUUCAAGGUAUCUGUU | C | 253.98 (S.D. 68.09) |
| (SEQ ID NO: 41) | | |
| UAAUAGGGCACUUGCACACUU | A | 220.75 (S.D. 30.82) |
| (SEQ ID NO: 42) | | |
| UUAUAGCAGUAUCUCCAGCUU | B | 286.02 (S.D. 23.99) |
| (SEQ ID NO: 43) | | |
| UUACUGGGCGCCUACCCCUUU | A | 268.24 (S.D. 81.60) |
| (SEQ ID NO: 44) | | |
| UGUUUGUGGUUUCCUACUCUU | A | 246.82 (S.D. 27.55) |
| (SEQ ID NO: 45) | | |
| UUUAUAGCAGUAUCUCCAGUU | B | 226.10 (S.D. 40.05) |
| (SEQ ID NO: 46) | | |
| AGUACUGAGCUCCUAAUUCUU | A | 224.47 (S.D. 18.46) |
| (SEQ ID NO: 47) | | |
| UUUUGUUCAAGGUAUCUGGUU | C | 222.90 (S.D. 35.56) |
| (SEQ ID NO: 48) | | |
| UUAAGCAAGCAGAUGCCAGUU | D | 261.23 (S.D. 29.76) |
| (SEQ ID NO: 49) | | |
| UUCAAAUUACACCAUUACUUU | B | 219.94; (S.D. 12.77) |
| (SEQ ID NO: 50) | | |
| CUACUAUGUUAUAAACUUAUU | A | 207.15; (S.D. 25.35) |
| (SEQ ID NO: 51) | | |
| GGAUUUUUGUUCAAGGUAUUU | C | 207.50; (S.D. 23.33) |
| (SEQ ID NO: 52) | | |
| GAAUACACGAAUAGAGCAAUU | A | 206.65; (S.D. 28.53) |
| (SEQ ID NO: 53) | | |
| CUCCAUGCUUCUCGAGUUAUU | A | 229.06; (S.D. 15.86) |
| (SEQ ID NO: 54) | | |
| AGUCUUCAGCGCUUCUCUAUU | C | 220.75; (S.D. 53.12) |
| (SEQ ID NO: 55) | | |
| GGAAUACACGAAUAGAGCAUU | A | 214.91; (S.D. 33.68) |
| (SEQ ID NO: 56) | | |
| GUCUGAAGGCUUUAGUAAAUU | C | 224.77; (S.D. 28.82) |
| (SEQ ID NO: 57) | | |
| CCUACUCCAUGCUUCUCGAUU | D | 198.15; (S.D. 21.93) |
| (SEQ ID NO: 58) | | |
| ACGGCUUCCCGUGAAUCAAUU | D | 206.01; (S.D. 55.01) |
| (SEQ ID NO: 59) | | |
| CCUACUAUGUUAUAAACUUUU | D | 238.46; (S.D. 12.52) |
| (SEQ ID NO: 60) | | |
| AAUUACAAGGCAGUCCCUUUU | B | 243.41; (S.D. 34.58) |
| (SEQ ID NO: 61) | | |
| UAAUGCUCCUACUAUGUUAUU | D | 274.75; (S.D. 43.59) |
| (SEQ ID NO: 62) | | |
| ACACUCAUUCAAAUUACACUU | B | 224.92; (S.D. 60.94) |
| (SEQ ID NO: 63) | | |
| GUCUUCAGCGCUUCUCUACUU | C | 232.21; (S.D. 29.07) |
| (SEQ ID NO: 64) | | |

Expression levels of ADAR1 p150 mRNA and protein in HELA cells, transfected with ADAR1 p150 aRNA are assessed and compared to control ADAR1 p150 levels of HELA cells not transfected with ADAR1 p150 aRNA, substantially as described herein. Briefly, BIELA cells are seeded 10,000 cells/well on 96-well plate setting in serum-free medium (Opti-MEM, Catalog #11058021). Thereafter, cells are either treated with 100 nM of an ADAR p150 aRNA (antisense sequence as set forth in Table 6b) with vehicles (Lipofectamine RNAiMax, Catalog #13778100) or with vehicles alone as controls (not transfected with an ADAR p150 aRNA) for no more than 12 hours. Media on treated and untreated cells are changed to full media (DMEM+10% FBS) 8-12 hours post-transfection to maintain cell culture until endpoint. ADAR1 p150 mRNA levels are measured using quantitative RT-PCR (TaqMan™ Fast Advanced Master Mix, Catalog #4444965) from cDNA synthesized according to manufacturer's instructions (SYBR Green Fast Advanced Cells-to-cT Kit, Catalog #A35379). As demonstrated in Table 6b, three specific ADAR1 p150 Targeting Regions (denoted Region A, B, and C in Table 5) are identified and ADAR1 p150 aRNA antisense sequences targeting one of those three regions demonstrate an increase of up to 350% ADAR1 p150 transcription.

TABLE 6b
Exemplified ADAR1 p150 aRNA Antisense
Sequences and % Increase in ADAR1
Transcription Expression:

| RNA antisense | ADAR1_p150 | % Increase in |
|---|---|---|
| sequence | Region | ADARI expression |
| AAGGCAGUCCCUUUAUAGCUU | C | 486.9; S.D. (100.8) |
| (SEQ ID NO: 37) | | |
| UAUAGCAGUAUCUCCAGCUUU | C | 185.5; S.D. (19.5) |
| (SEQ ID NO: 38) | | |
| AACUUAAGCAAGCAGAUGCUU | A | 305.3; S.D. (39.8) |
| (SEQ ID NO: 108) | | |
| UUAUAGCAGUAUCUCCAGCUU | C | 223.9; S.D. (51.2) |
| (SEQ ID NO: 43) | | |
| UGGUUUCCUACUCCAUGCUUU | D | 864.8; S.D. (231.1) |
| (SEQ ID NO: 66) | | |
| UUUUGUUCAAGGUAUCUGGUU | B | 272.3; S.D. (49.7) |
| (SEQ ID NO: 48) | | |
| GGAUUUUUGUUCAAGGUAUUU | B | 212.3; S.D. (23.1) |
| (SEQ ID NO: 52) | | |
| GCUUCUCUACACUCAUUCAUU | B | 254.2; S.D. (121.8) |
| (SEQ ID NO: 109) | | |
| AGAUCUCGUCAAACGACCAUU | A | 306.4; S.D. (57.9) |
| (SEQ ID NO: 110) | | |
| CCUACUAUGUUAUAAACUUUU | A | 229.2; S.D. (75.4) |
| (SEQ ID NO: 60) | | |
| CCAUGCUUCUCGAGUUACUUU | D | 487.1; S.D. (121.9) |
| (SEQ ID NO: 111) | | |
| ACUGAGCUCCUAAUUCCCCUU | A | 230.3; S.D. (48.0) |
| (SEQ ID NO: 112) | | |
| CAUUCAAAUUACACCAUUAUU | B | 412.5; S.D. (88.4) |
| (SEQ ID NO: 113) | | |

ADAR1 P110 protein levels are measured using semi-quantitative of Western blotting. Percentage of ADAR1 protein in HELA cells transfected with 100 nM and 10 nM of exemplified ADAR aRNA comprising sense of SEQ TD NO. 65 and antisense of SEQ ID NO. 66 are provided in Table 6c. ADAR1 protein in HELA cells transfected with 100 nM and 10 nM of nontargeting aRNA is also provided; the nontargeting aRNA comprising a sense strand (UCCUAUGACUGUAGAUUUUAU SEQ TD NO: 135) and an antisense strand (AUAAAAUCUACAGUCAUAGGAAU SEQ TD NO: 136). ADAR1 protein levels in HELA cells treated with recombinant IFN-beta for 24 hours is also provided as a positive control. Results shown as percentage of ADAR1 protein compared to untreated HELA cells.

TABLE 6c
% Increase in ADAR1 Protein vs Untreated HELA Cells:

| | % ADAR1 Protein |
|---|---|
| Treatment Group | vs. Untreated |
| ADAR1 aRNA (sense SEQ ID NO. 65; | 301.50% |
| antisense SEQ ID NO. 66) - 100 nM | |
| ADAR1 aRNA (sense SEQ ID NO. 65; | 228.05% |
| antisense SEQ ID NO. 66) - 10 nM | |
| Non-targeting aRNA - 100 nM | 93.90% |
| Non-targeting aRNA - 10 nM | 93.53% |
| Positive Control | 346.19% |
| Untreated | 100.00% |

### Example 2C. ADAR2

Expression levels of ADAR2 mRNA and protein in Hela cells transfected with ADAR2 aRNA are assessed and compared to control ADAR2 levels of Hela cells not transfected with ADAR2 aRNA, substantially as described herein. Briefly, Hela cells are seeded 10,000 cells/well on 96-well plate setting in serum-free medium (Opti-MEM, Catalog #11058021). Thereafter, cells are either treated with 100 nM of an ADAR2 aRNA (antisense sequence as set forth in Table 8) with vehicles (Lipofectamine RNAiMax, Catalog #13778100) or with vehicles alone as controls (not transfected with an ADAR2 aRNA) for no more than 12 hours. Media on treated and untreated cells are changed to full media (DMEM+10% FBS) 8-12 hours post-transfection to maintain cell culture until endpoint. ADAR2 mRNA levels are measured using quantitative RT-PCR (TaqMan™ Fast Advanced Master Mix, Catalog #4444965) from cDNA synthesized according to manufacturer's instructions (SYBR Green Fast Advanced Cells-to-cT Kit, Catalog #A35379). As demonstrated in Table 8, three specific ADAR2 Targeting Regions (denoted Region A, B, C and D in Table 7) are identified and ADAR2 aRNA antisense sequences targeting one of those three regions demonstrate an increase of up to 200% ADAR2 transcription.

TABLE 7
ADAR2 Targeting Regions:

| | |
|---|---|
| | Region −801 to −500 |
| Region A | (Reversed to 5′-3′) |

(SEQ ID NO: 8)
ACCACCATCACCTAAACGTTGGTAACTGGAGCAGCTGCTAGTGTCAGTGC
GGACTAAACAGGAGACGGCGGGAACCGTGTCCAGCCAGGGTCCTGGGCCG
CGACCTGGTTCTCCCGGAGTCTACAGTGAGGATGACGGGCGGGGAGAGGG
GGCCGGCGGGACCCGCGTGTCCCAGGCAACTCCGGGAGAGGGAGAAGCAG
GGGTGGCTCGGCGGGGGCTCGGCGGGGGCTCGGCGGGGGCTCGGCGGGGG
CTCGGCGGGGGCTCGGCGGGGGCTCGGCGGGGGCAGCGCCCGCTGCAGGG
AG

| | |
|---|---|
| | Region −1324 to −835 |
| Region B | (Reversed 5′-3′) |

(SEQ ID NO: 9)
CCCAGCCCCAGCCTCCAGACTGGCTGAGATCACAGTGTGCGCCACCCCAT
CCCTGGCTTGCGATGGTCTCTTACTCCCCACTTTACAAGGGAGGAAAGAG
GCCGAGGGCAGAGTGGGCCGCCTGAGGTTTGGAGGCCAGGGCTAGTACAA
CCTCAATTTGACCCTGGAACCTGCCGCTTCCCCCACCCAGGTGCGGGACC
CACCCGCTTGTCCACACCTGGCTCTGCCCACCGCCCCCGGCTGCTCCTCT
GGGCTCAGGTCGCCGCGGTCAGGAGCTGCCAAGTTTGCCTATCAAACTTT
ATCTTCGTGCAGAGAACTGCAGCCTGGAGCTGGTTATTCCGGTCAGTGAA
AACGTTGCATTTCTACATATGCTTATCATCATCTGTGTAAACATTTCTTG
GTATAACTGTGGAACAGTCAGTAAATATAGATAAATCGAAGAGTAGGTCT
ATTGCATATGCTATAAAAAAATGCTTTTACTATCAACCTA

| | |
|---|---|
| | Region −1773 to −1566 |
| Region C | (Reversed 5′-3′) |

(SEQ ID NO: 10)
CACACAACGTCCTCAGGCACGTATCTCTTTAAGAATGTGTCCCGAGAAGG
CGTCCCAGGAGTCTTGATTTTTATTTAGCCGTCCACGGTTGCCCCTTTGG
GTGCTTCGCCTTCAGATGGGGTGAAGGGCTCACTTGTTAGCTGGCTGGCC
CCAAAGACAGGCTTGTTTTCCACCAGGCAGTGACTGAAGCCGGCAGCCTG
CTGCATAC

| | |
|---|---|
| | Region −3000 to −1924 |
| Region D | (Reversed 5′-3′) |

(SEQ ID NO: 11)
ATTTAACTGCACTGAAGGGAAAACGTGGGAAATGGATTTTTGGTGCTGTG
TAGACCACATTTCATAGCGGTTGGCATCTCACATGCTTATGCAAAGCCTA
ACTCCGCACCCTGGGGCAGACAGTGGGAGCCCAGCTGGATTCCTACACTC
AAGCCCTCCAGCATCAGGTTTATTTTCCAGGACACCAGAGTGATTGTTTA
TTCCATAATTCCCACAAAGGAGACAGTAAACAACAGAACAGAGGTGGAGC
GGGCACGGAAGGCCAGGAAGTGGATGGTGGCTGCCAGCACTTCTGTGGAG
ACCCAGGGCCCCCCTCCAGGAGCCCCGGTGTTCAACCTCCACAGTGAACA
GGGGATGGATGGCTGAGATGTCCTCGAAATTTTTTTGGACTTCCTCAGGC
GACCACAGGATTCCTTCTCAAAGAGCCTGATCTCAGCAGGCACCCGAATG
GGCATCCTGGTGCTTCATGCTCTACAACAGCTGGGAACGCCATGTCCTGG
CCCCAGGCGACTGGAAACTGCTTTCCTCCCCGACATCAGCACCAAGGGGA
ATGTTCCCAGTGCCATTCTTCCAAGTCAGGGAGAGCGTCACAATAGAAAC
CGTCTCTGTGGAGAGGATGGCACCTGAAGCCATGGAATAGGAAGGGAGCA
TCAGAGGCTGCTGGCTGGTCCTGCAGAGCCGCCTGAGAGGCCTGTGGGAG
CAGCAGAGGGTCCCGGCCTTGGGCGCCATCCGCTCTCTGCTGCTCTGGAG
GGAGAAAAAGGACAAGTTGAAACTTGCACAAGCAGCCTCCATTCTGGGGA
GTTCCCTTGTATTCCCCACACCAACCCGCACCTCAGCGAAGGCCTGTGGA
AGACTTCTGCAGTGACAGCCCCGATGAACCATGCTTGCCGTGCCCGTCCC
CTGTGCGGTGCCTCACGTCCACTCAGGCCCCGGCCATCTCACCCTCCTGG
GGAGATGGAGGGAAGCACCATGGGGATTTGCTTTTTCTTGCTGCCGACGG
AGCCCAGCCACCACGGGAGGGAGGCCCGGCCAGCCTGCGGTGGGTGGGTG
ACATGTGGCCCGGATCTGCCCGGGGCG

TABLE 8
Exemplified ADAR2 aRNA Antisense Sequences and
% Increase in ADAR1 Transcription Expression:

| RNA antisense | ADAR2 | % Increase in |
|---|---|---|
| sequence | Region | ADAR2 expression |
| AAGUGGGGAGUAAGAGACCUU | B | 163.6; S.D. (18.7) |
| (SEQ ID NO: 114) | | |
| AUCGCAAGCCAGGGAUGGGUU | B | 148.5; S.D. (6.2) |
| (SEQ ID NO: 115) | | |
| ACCUGGGUGGGGGAAGCGGUU | B | 191.7; S.D. (43.2) |
| (SEQ ID NO: 116) | | |
| ACUCUGCCCUCGGCCUCUUUU | B | 176.7; S.D. (21.5) |
| (SEQ ID NO: 117) | | |
| ACUAGCCCUGGCCUCCAAAUU | B | 171.8; S.D. (10.9) |
| (SEQ ID NO: 118) | | |
| AUGGGGUGGCGCACACUGUUU | B | 225.4; S.D. (16.7) |
| (SEQ ID NO: 119) | | |
| CACUCUGCCCUCGGCCUCUUU | B | 207.2; S.D. (9.8) |
| (SEQ ID NO: 120) | | |
| CAGGUGUGGACAAGCGGGUUU | B | 151.4; S.D. (12.6) |
| (SEQ ID NO: 121) | | |
| CCCUCGGCCUCUUUCCUCCUU | B | 158.6; S.D. (17.0) |
| (SEQ ID NO: 122) | | |
| CCUGGCCUCCAAACCUCAGUU | B | 164.3; S.D. (3.9) |
| (SEQ ID NO: 123) | | |
| CUCGGCCUCUUUCCUCCCUUU | B | 192.2; S.D. (7.0) |
| (SEQ ID NO: 124) | | |
| CUGGCCUCCAAACCUCAGGUU | B | 163.0; S.D. (56.3) |
| (SEQ ID NO: 125) | | |
| GGCCUCCAAACCUCAGGCGUU | B | 163.5; S.D. (10.8) |
| (SEQ ID NO: 126) | | |
| GGUGGGCAGAGCCAGGUGUUU | B | 52.3; S.D. (2.5) |
| (SEQ ID NO: 127) | | |
| GGUGUGGACAAGCGGGUGGUU | B | 173.6; S.D. (21.8) |
| (SEQ ID NO: 128) | | |
| GUAAAGUGGGGAGUAAGAGUU | B | 149.2; S.D. (9.2) |
| (SEQ ID NO: 129) | | |
| GUACUAGCCCUGGCCUCCAUU | B | 270.5; S.D. (17.1) |
| (SEQ ID NO: 130) | | |
| GUGGACAAGCGGGUGGGUCUU | B | 188.0; S.D. (5.8) |
| (SEQ ID NO: 131) | | |
| GUGGGGAGUAAGAGACCAUUU | B | 178.8; S.D. (4.3) |
| (SEQ ID NO: 132) | | |
| GUGGGGGAAGCGGCAGGUUUU | B | 45.5; S.D. (4.1) |
| (SEQ ID NO: 133) | | |

### Example 3: ADAR-Catalyzed RNA Editing

Assessment of ADAR aRNA candidates in RNA editing therapy may be assessed substantially as described herein. ADAR aRNA candidates may be co-delivered with therapeutic RNA (e.g., guide RNA) designed for editing target RNA. This may be carried out in vitro and/or in vivo. For in vitro assay, a serial concentration of ADAR aRNA and therapeutic RNA may be transfected into target cell line for 24, 48 and 72 hours. Additionally, ADAR aRNA and therapeutic RNA may be linked to or encapsulated in a delivery vehicle (e.g. GalNAc conjugation, liposome, etc.).

Following transfection, cell lysate may then be processed to perform target transcript analysis by RNA sequencing technology. The ratio of edited target RNA transcript is then able to be used to evaluate the RNA editing efficacy (with comparison against control groups, including control groups of ADAR aRNA or therapeutic RNA only transfected cells).

Editing efficacy of a therapeutic RNA is evaluated in vitro substantially as described herein. Briefly, Hela cells are seeded 10,000 cells/well on 96-well plate in serum-free medium (Opti-MEM, Catalog #11058021). Cells are transfected with 100 nM of therapeutic RNA (ASO targeting either beta-actin gene or GAPDH gene). Cells transfected with therapeutic RNA are co-treated for no more than 12 hours with either: (i) 100 nM of an ADAR p150 aRNA (antisense sequence as set forth in Table 9) with vehicles (Lipofectamine RNAiMax, Catalog #13778100); or (ii) vehicles alone. Media is changed to full media (DMEM+10% FBS) 8-12 hours post-transfection. ADAR1 mRNA levels are measured using quantitative RT-PCR (TaqMan™ Fast Advanced Master Mix, Catalog #4444965) from cDNA synthesized according to manufacturer's instructions (SYBR Green Fast Advanced Cells-to-cT Kit, Catalog #A35379). Editing efficiency is assessed by Sanger sequencing (i.e., to quantify the A to G editing efficacy). As demonstrated in Table 9a, co-transfection of therapeutic RNA for beta actin gene, with ADAR1 p150 aRNA, demonstrates an increase of up to 600% ADAR1 transcription and editing efficacy enhancement of up to 350%. As demonstrated in Table 9b, co-transfection of therapeutic RNA for GAPDH gene, with ADAR1 p150 aRNA, demonstrates an increase of up to 1000% ADAR1 transcription and editing efficacy enhancement of up to 150%.

TABLE 9
Editing Efficiency of beta actin with
ADAR aRNA Co-transfection:

| ADAR1 p150 aRNA | ADAR1 | % increase | | |
|---|---|---|---|---|
| sequence (5′->3′) | p150 | in ADAR | % | % |
| Editing Efficiency | Region | expression | editing | improvement* |
| Sense: | C | 665.6; SD | 22.6 SD | 330.2 SD |
| GCUAUAAAGGGACUGCCUUUU | | (108.2) | (7.1) | (104.2) |
| (SEQ ID NO: 79) | | | | |
| Antisense: | | | | |
| AAGGCAGUCCCUUUAUAGCUU | | | | |
| (SEQ ID NO: 37) | | | | |
| Sense: | A | 369.1; SD | 25.5 SD | 372.3 SD |
| GCAUCUGCUUGCUUAAGUUUU | | (52.4) | (10.1) | (146.8) |
| (SEQ ID NO: 134) | | | | |
| Antisense: | | | | |
| AACUUAAGCAAGCAGAUGCUU | | | | |
| (SEQ ID NO: 108) | | | | |
| Sense: | D | 642.5; SD | 22.7 SD | 330.9 SD |
| AGCAUGGAGUAGGAAACCAUU | | (196) | (10.4) | (151.5) |
| (SEQ ID NO: 65) | | | | |
| Antisense: | | | | |
| UGGUUUCCUACUCCAUGCUUU | | | | |
| (SEQ ID NO: 66) | | | | |
| Neg. control | NA | 151.5; SD | 6.8 SD | NA |
| | | (24.0) | (6.9) | |

*% improvement = (% editing of treated group)/(% editing of Neg. Control)

TABLE 9b
Editing Efficiency of GAPDH with
ADAR aRNA Co-transfection:

| | | | Editing | |
|---|---|---|---|---|
| | ADAR1 | % increase | Efficiency | |
| ADAR1 p150 aRNA | p150 | in ADAR | % | % |
| sequence (5′->3′) | Region | expression | editing | improvement* |
| Sense: | C | 393.0; SD | 86.2 SD | 156.6; SD |
| GCUAUAAAGGGACUGCCUUUU | | (48.3) | (1.3) | (2.4) |
| (SEQ ID NO: 79) | | | | |
| Antisense: | | | | |
| AAGGCAGUCCCUUUAUAGCUU | | | | |
| (SEQ ID NO: 37) | | | | |
| Sense: | A | 461.1; SD | 77.1 SD | 140.0; SD |
| GCAUCUGCUUGCUUAAGUUUU | | (74.5) | (2.0) | (3.6) |
| (SEQ ID NO: 134) | | | | |
| Antisense: | | | | |
| AACUUAAGCAAGCAGAUGCUU | | | | |
| (SEQ ID NO: 108) | | | | |
| Sense: | D | 1368.7; SD | 85.4 SD | 155.3; SD |
| AGCAUGGAGUAGGAAACCAUU | | (97.6) | (0.4) | (0.7) |
| (SEQ ID NO: 65) | | | | |
| Antisense | | | | |
| UGGUUUCCUACUCCAUGCUUU | | | | |
| (SEQ ID NO: 66) | | | | |
| Neg. control | NA | 161.9; SD | 58.0 SD | 105.5; SD |
| | | (18.3) | (1.4) | (2.6) |

*% improvement = (% editing of treated group)/(% editing of Neg. Control)

### Example 4: ADAR3 Expression Modulation

Embodiments of the present disclosure provide ADAR3 aRNA for upregulation of endogenous ADAR3. In specific embodiments, ADAR3 aRNA is delivered to tissues for the treatment of diseases associated with ADAR1-catalyzed or ADAR2-catalyzed hyperactive transcript editing. Accordingly, upregulation of endogenous expression of ADAR3 may regulate, for example, through competitive inhibition, reduced binding efficiency, reduced activity and/or reduced expression, ADAR1 and/or ADAR2 activity, thereby regulating ADAR1-catalyzed or ADAR2-catalyzed hyperactive transcript editing. In some specific embodiments, ADAR3 aRNA is co-delivered with therapeutic RNA designed for editing target RNA for providing a therapeutic benefit in diseases associated with ADAR1-catalyzed or ADAR2-catalyzed hyperactive transcript editing.

Briefly, ADAR3 aRNA, according to methods provided herein, is prepared in relation to the ADAR3 aRNA target sequences provided by SEQ ID NO: 12 and/or 13 and may be prepared substantially as described in Example 1. In-vitro ADAR3 expression, both baseline and post-ADAR3 aRNA transfection, may be assessed using methods substantially as described herein and at Example 2. Additionally, co-delivery of ADAR3 aRNA and therapeutic RNA designed for editing target RNA, for providing a therapeutic benefit in diseases associated with ADAR1-catalyzed or ADAR2-catalyzed hyperactive transcript editing, may be assessed substantially as described herein and at Example 3.

According to specific embodiments, ADAR3 aRNA upregulation of endogenous ADAR3 is delivered to central nervous system tissue (with or without therapeutic RNA designed for editing target RNA) for regulation of ADAR1-catalyzed or ADAR2-catalyzed hyperactive transcript editing. In more specific embodiments, ADAR3 aRNA upregulation of endogenous ADAR3 is delivered to central nervous system tissue for regulation of ADAR1-catalyzed or ADAR2-catalyzed hyperactive transcript editing associated with brain cancer (including glioblastoma), tumorigenesis and/or chronic neurological disorders. According to some embodiments ADAR3 aRNA upregulation of endogenous ADAR3 is delivered (with or without therapeutic RNA designed for editing target RNA) to non-CNS tissue (e.g., peripheral tissue) for regulation of ADAR1-catalyzed or ADAR2-catalyzed hyperactive transcript editing diseases, including oncogenesis, metastasis, immune and autoimmune disease (for example, systemic lupus erythematosus).

### Exemplary Embodiments

1. An adenosine deaminase acting on RNA enzyme (ADAR) activating RNA (aRNA) which upregulates expression of ADAR, wherein the ADAR aRNA comprises an antisense oligonucleotide sequence.
2. The ADAR aRNA of embodiment 1, wherein ADAR is ADAR1p110.
3. The ADAR aRNA of embodiment 1, wherein ADAR is ADAR1p150.
4. The ADAR aRNA of embodiment 1, wherein ADAR is ADAR2.
5. The ADAR aRNA of embodiment 1, wherein ADAR is ADAR3.
6. The ADAR aRNA of any of embodiments 1-5, wherein the antisense oligonucleotide sequence is approximately 15 to approximately 50 nucleotides.
7. The ADAR aRNA of any of embodiments 1-5, wherein the antisense oligonucleotide sequence is approximately 19 to approximately 30 nucleotides.
8. The ADAR aRNA of any of embodiments 1-7, wherein the ADAR aRNA further comprises a sense oligonucleotide sequence.
9. The ADAR aRNA of embodiment 8, wherein the antisense sequence is at least 80% complementary to a target sequence.
10. The ADAR aRNA of embodiment 9, wherein the target sequence is within −3000 to +150 nucleotides of the ADAR target sequence transcription start site.
11. The ADAR aRNA of embodiment 9, wherein the target sequence is within SEQ ID NOs: 1, 2, 3, 4, 5, 6, 7, 8, 9, 10, 11, 12 and/or 13.
12. The ADAR aRNA of any of embodiments 1-11, wherein at least one of the antisense and sense oligonucleotide sequence comprises a 3′ tail.
13. The ADAR aRNA of any of embodiments 1-12, wherein at least one of the antisense and sense oligonucleotide sequence comprises at least one modified nucleotide.
14. The ADAR aRNA of embodiment 13, wherein the at least one modified nucleotide comprises a nucleotide modification from at least one of a thio-modified, an amino-modified, a phosphate-modified, a cholesterol-triethylene glycol (TEG)-modified, a methyl-modified, and a fluoro-modified nucleotide.
15. The ADAR aRNA of any of embodiments 1-14, wherein the antisense and sense oligonucleotide sequences are each independently approximately 15 to approximately 50 nucleotides.
16. The ADAR aRNA of any of embodiments 1-14, wherein the antisense and sense oligonucleotide sequences are each independently approximately 19 to approximately 30 nucleotides.
17. The ADAR aRNA of any of embodiments 1-14, wherein the antisense and sense oligonucleotide sequences are each 22 nucleotides.
18. The ADAR aRNA of any of embodiments 1-14, wherein the antisense and sense oligonucleotide sequences are each 21 nucleotides.
19. The ADAR aRNA of any of embodiments 1-18 wherein at least one of the antisense and sense oligonucleotide sequences comprises a 3′ overhang.
20. The ADAR aRNA of any of embodiments 1-19, wherein at least one of the antisense and sense oligonucleotide sequences is comprised on a nucleic acid vector.
21. The ADAR aRNA of any of embodiments 1-20, wherein the aRNA is linked to a ligand targeting moiety.
22. The ADAR aRNA of embodiment 21, wherein the ligand targeting moiety is GalNAc.
23. The ADAR aRNA of any of embodiments 1-22, wherein the aRNA is linked to a second RNA.
24. The ADAR aRNA of embodiment 23, wherein the second RNA is a therapeutic RNA.
25. The ADAR aRNA of embodiment 24, wherein the therapeutic RNA comprises one of: an mRNA; miRNA; sgRNA; aRNA; iRNA; or ASO.
26. The ADAR aRNA of any of embodiments 1-25, wherein the aRNA binds AGO2 protein.
27. The ADAR aRNA of any of embodiments 1-26, wherein the aRNA is linked to a delivery vehicle.
28. The ADAR aRNA of embodiment 27, wherein the delivery vehicle comprises one of: an antibody, or fragment thereof; a scFv; a peptide; GalNAc; an apatamer; or a nanoparticle.
29. The ADAR aRNA of any of embodiments 1-27, wherein the aRNA is encapsulated, fully or partially, within a delivery vehicle.
30. The ADAR aRNA of embodiment 29, wherein the delivery vehicle comprises one of: a lipidoid; liposome; lipoplex; polymer; or nanoparticle.
31. The ADAR aRNA of any of embodiments 1-4 and 6-30, wherein the antisense oligonucleotide sequence comprises at least one of SEQ ID NOs. 14-134.
32. An ADAR1p110 aRNA comprising an antisense oligonucleotide sequence given by one of SEQ ID NOs. 14-36 and 100-107.
33. An ADAR1p150 aRNA comprising an antisense oligonucleotide sequence given by one of SEQ ID NOs. 37-99, 108-113 and 134.
34. An ADAR2 aRNA comprising an antisense oligonucleotide sequence given by one of SEQ ID NOs. 114-133.
35. A method of modulating expression of ADAR comprising administering to a patient the ADAR aRNA of any of embodiments 1-34, 60 and 74-76.
36. The method of embodiment 35, wherein ADAR expression is increased.
37. The method of embodiment 36, wherein ADAR expression is increased by at least 20%.
38. The method of embodiment 36, wherein ADAR expression is increased by at least 30%.
39. The method of embodiment 36, wherein ADAR expression is increased by at least 40%,
40. The method of embodiment 36, wherein ADAR expression is increased by at least 50%,
41. An RNA editing therapeutic comprising:
   a therapeutic RNA; and
   an ADAR aRNA of any of embodiments 1-34, 60 and 74-76.
42. The RNA editing therapeutic of embodiment 41, wherein the therapeutic RNA comprises one of an: mRNA; miRNA; sgRNA; aRNA; iRNA; or ASO.
43. A method of treating a disease in human comprising administering a therapeutically effective amount of an RNA editing therapeutic to the human, wherein the RNA therapeutic comprises: a therapeutic RNA; and an ADAR aRNA of any of embodiments 1-34, 60 and 74-76.
44. The method of embodiment 43, wherein the therapeutic RNA and the ADAR aRNA are co-administered.
45. The method of embodiment 43, wherein the therapeutic RNA and the ADAR aRNA are co-formulated.
46. The method of embodiment 43, wherein the therapeutic RNA and the ADAR aRNA are linked.
47. The method of any of embodiments 43-46, wherein at least one of the therapeutic RNA and the ADAR aRNA are encapsulated, fully or partially, within a delivery vehicle.
48. The method of embodiment 47, wherein the delivery vehicle comprises one of: a lipid; liposome; lipoplex; polymer; or nanoparticle.
49. The method of any of embodiments 43-46, wherein at least one of the therapeutic RNA and the ADAR aRNA are linked to a delivery vehicle.
50. The method of embodiment 49, wherein the delivery vehicle comprises one of: an antibody, or fragment thereof, a scFv; a peptide; GalNAc; an apatamer; or a nanoparticle.
51. A pharmaceutical composition comprising:
   a therapeutic RNA;
   an ADAR aRNA of any of embodiments 1-34, 60 and 74-76; and
   at least one pharmaceutically acceptable excipient.
52. The pharmaceutical composition of embodiment 51, wherein the therapeutic RNA comprises one of an: mRNA; miRNA; sgRNA; aRNA; iRNA; or ASO.
53. A method of treating a disease in human comprising administering a therapeutically effective amount of a pharmaceutical composition the human, wherein the pharmaceutical composition comprises: a therapeutic RNA, an ADAR aRNA of any of embodiments 1-34, 60 and 74-76, and a pharmaceutically acceptable excipient.
54. The method of embodiment 53, wherein the therapeutic RNA and the ADAR aRNA are co-administered.
55. The method of embodiment 53, wherein the therapeutic RNA and the ADAR aRNA are co-formulated.
56. The method of embodiment 53, wherein the therapeutic RNA and the ADAR aRNA are linked.
57. The method of any of embodiments 53-56, wherein at least one of the therapeutic RNA and the ADAR aRNA are encapsulated, fully or partially, within a delivery vehicle.
58. The method of embodiment 57, wherein the delivery vehicle comprises one of: a lipidoid; liposome; lipoplex; polymer; or nanoparticle.
59. The method of any of embodiments 53-58, wherein at least one of the therapeutic RNA and the ADAR aRNA are linked to a delivery vehicle.
60. The method of embodiment 59, wherein the delivery vehicle comprises one of: an antibody, or fragment thereof, a scFv; a peptide; GalNAc; an apatamer; or a nanoparticle.
61. The ADAR aRNA of any of embodiments 5-25 and 27-30, wherein the ADAR is ADAR3 and the target sequence is within SEQ ID NOs: 12 and/or 13.
62. A method of treating a disease in a human comprising administering a therapeutically effective amount of an ADAR3 aRNA of embodiment 61 to the human.
63. The method of embodiment 62, wherein the ADAR3 aRNA is delivered to a tissue exhibiting overexpression of ADAR1 and/or ADAR2.
64. The method of embodiment 62 or 63, wherein the ADAR3 aRNA is delivered to the CNS.
65. The method of any of embodiments 62-64, wherein the ADAR3 aRNA is encapsulated, fully or partially, within a delivery vehicle.
66. The method of embodiment 65, wherein the delivery vehicle comprises one of: a lipid; liposome; lipoplex; polymer; or nanoparticle.
67. The method of any of embodiments 62-66, wherein the ADAR3 aRNA is linked to a delivery vehicle.
68. The method of any of embodiments 65-67, wherein the delivery vehicle comprises one of: an antibody, or fragment thereof; a scFv; a peptide; GalNAc; an apatamer; or a nanoparticle.
69. The method of any of embodiments 62-68 further comprising the step of administering a therapeutically effective amount of a therapeutic RNA to the human.
70. The method of embodiment 69, wherein the therapeutic RNA and the ADAR3 aRNA are co-administered.
71. The method of embodiment 70, wherein the therapeutic RNA and the ADAR3 aRNA are co-formulated.
72. The method of embodiment 70 or 71, wherein the therapeutic RNA and the ADAR3 aRNA are linked.
73. The method of any of embodiments 62-72, wherein the disease is characterized by ADAR1-catalyzed or ADAR2-catalyzed hyperactive transcript editing.
74. The method of any of embodiments 62-72, wherein the disease is one of cancer, tumorigenesis, metastasis, brain cancer including glioblastoma, a chronic neurological disorder, an immune disease or an autoimmune disease including systemic lupus erythematosus.
75. The ADAR aRNA of any of embodiments 2, 6-30, and 32 wherein the ADAR is ADAR1p110 and the target sequence is within SEQ ID NOs: 1, 2 and/or 3.
76. The ADAR aRNA of any of embodiments 3, 6-30 and 33, wherein the ADAR is ADAR1p150 and the target sequence is within SEQ ID NOs: 4, 5, 6 and/or 7.
77. The ADAR aRNA of any of embodiments 4 and 6-30, wherein the ADAR is ADAR2 and the target sequence is within SEQ ID NOs: 8, 9, 10 and/or 11.

1. An adenosine deaminase acting on RNA enzyme (ADAR) activating RNA (aRNA) that upregulates expression of ADAR, wherein the ADAR aRNA comprises an antisense oligonucleotide sequence, and wherein the target sequence is within SEQ ID NOs: 1, 2, 3, 4, 5, 6, 7, 8, 9, 10, 11, 12 or 13.

2. The ADAR aRNA of claim 1, wherein ADAR is ADAR1p110, ADAR1p150, ADAR2 or ADAR3.

3. The ADAR aRNA of claim 1, wherein the antisense oligonucleotide sequence is approximately 15 to approximately 50 nucleotides.

4. The ADAR aRNA of claim 1, wherein the antisense oligonucleotide sequence is approximately 19 to approximately 30 nucleotides.

5. The ADAR aRNA of claim 1, wherein the ADAR aRNA further comprises a sense oligonucleotide sequence.

6. The ADAR aRNA of claim 5, wherein the antisense sequence is at least 80% complementary to a target sequence.

7. The ADAR aRNA of claim 6, wherein the target sequence is within −3000 to +150 nucleotides of the ADAR target sequence transcription start site.

8. (canceled)

9. The ADAR aRNA of claim 1, wherein at least one of the antisense and sense oligonucleotide sequence comprises at least one modified nucleotide, wherein the at least one modified nucleotide comprises a nucleotide modification from at least one of a thio-modified, an amino-modified, a phosphate-modified, a cholesterol-triethylene glycol (TEG)-modified, a methyl-modified, and a fluoro-modified nucleotide.

10. The ADAR aRNA of claim 1, wherein the antisense and sense oligonucleotide sequences are each independently approximately 19 to approximately 30 nucleotides.

11. The ADAR aRNA of claim 10, wherein the antisense and sense oligonucleotide sequences are each 21 nucleotides or the antisense and sense oligonucleotide sequences are each 21 nucleotides.

12. The ADAR aRNA of claim 1, wherein at least one of the antisense and sense oligonucleotide sequences comprises a 3′ overhang.

13. The ADAR aRNA of claim 1, wherein the aRNA is linked to a therapeutic RNA.

14. The ADAR aRNA of claim 13, wherein the therapeutic RNA comprises one of an mRNA, miRNA, sgRNA, aRNA, iRNA or ASO.

15. The ADAR aRNA of claim 1, wherein the aRNA is linked to a delivery vehicle.

16. The ADAR aRNA of claim 15, wherein the delivery vehicle comprises one of an antibody or fragment thereof, a scFv, a peptide, GalNAc, an apatamer+ or a nanoparticle.

17. The ADAR aRNA of claim 15, wherein the aRNA is encapsulated, fully or partially, within a delivery vehicle, wherein the delivery vehicle comprises one of a lipidoid, liposome, lipoplex, polymer or nanoparticle.

18. The ADAR aRNA of claim 1, wherein the antisense oligonucleotide sequence comprises at least one of SEQ ID NOs. 14-134.

19. An ADAR1p110 aRNA comprising an antisense oligonucleotide sequence given by one of SEQ ID NOs. 14-36 and 100-107.

20. An ADAR1p150 aRNA comprising an antisense oligonucleotide sequence given by one of SEQ ID NOs. 37-99, 108-113 and 134.

21. An ADAR2 aRNA comprising an antisense oligonucleotide sequence given by one of SEQ ID NOs. 114-133.

22. A method of modulating expression of ADAR comprising:
administering to a patient the ADAR aRNA of claim 1.

23. The method of claim 22, wherein ADAR expression is increased, by at least 20%, by at least 30%, by at least 40% or by at least 50%.

24. A method of treating a disease in human comprising:
administering a therapeutically effective amount of a therapeutic RNA to a human; and
administering an ADAR aRNA of claim 1 to the human.
